(19) United States
(12) Patent Application Publication
(10) Pub. No.: US 2019/0079092 A1
(43) Pub. Date: Mar. 14, 2019

(54) Method of Diagnosing Cancer by Determining the Level of One or More Volatile Organic Compounds

(71) Applicant: MDB Diagnosis Group Limited, London (GB)

(72) Inventors: Miles David Boyden, London (GB); Peter R.H. Jones, Bristol (GB)

(21) Appl. No.: 16/077,063

(22) PCT Filed: Feb. 8, 2017
(86) PCT No.: PCT/GB2017/050314

§ 371 Date: Aug. 10, 2018

(30) Foreign Application Priority Data
Feb. 10, 2016 (GB) 1602394.7

(51) Int. Cl. G01N 33/574; G01N 30/72

(52) U.S. Cl. CPC G01N 33/57488; G01N 2030/884; G01N 30/7206

(57) ABSTRACT

Disclosed is a method of diagnosing cancer in a subject, comprising determining a level of one or more volatile organic compounds for cancer in a urine sample obtained from the subject, wherein the identity and/or the level of the biomarker is determined by a mass spectrometry method, such as gas chromatography-mass spectrometry or tandem mass spectrometry. Specific biomarkers identified using the method and the use of the method to identify subjects as requiring treatment for cancers are also disclosed.

### FIELD OF THE INVENTION

This invention relates to a method of diagnosing cancer, and to biomarkers for various cancers identified by the method. It also relates to substances for use in treating cancers in subjects identified by the method.

### BACKGROUND TO THE INVENTION

Many cancers are diagnosed using biopsy methods. However, a biopsy is an invasive method which can be painful to the patient. There is therefore a need in the art for less invasive methods which do not cause suffering to the patient.

It is generally known in the art that volatile organic compounds (VOC's, as defined below) are present in human urine, and that volatile organic compounds may be biomarkers for certain cancers. For example, the existence of volatile bladder cancer biomarkers from human urine has been shown by sniffer dog studies, as described in Willis et al. Cancer Biomarkers 2011, 8, 145-153.

Previous studies analysing urine headspace volatile organic compounds from bladder cancer patients has shown a profile difference in volatile organic compounds between cancerous and non-cancerous (controls) urine samples. For example, Khalid et al., PLoS One, 2013, 8(7), 1-8, describes a pilot study combining a GC-sensor device with a statistical model for the identification of bladder cancer from urine headspace. However, although it is described therein that the technique allowed the differentiation of cancerous and non-cancerous (control) subjects, the studies failed to identify the specific volatile components.

Mass spectrometry (MS) is an analytical chemistry technique that helps identify the amount and type of chemicals present in a sample by measuring the mass-to-charge (m/z) ratio and abundance of gas-phase ions. The use of mass spectrometry techniques to identify compounds in urine samples obtained from a cancer patient is generally known in the art.

For example, Spanel et al. Rapid Commun. Mass Spectrom. 1999, 13, 1354, describes the use of mass spectrometry techniques to identify compounds in urine samples obtained from selected ion flow tube mass spectrometry (SIFT-MS). SIFT-MS uses precursor gas ions (e.g. water, nitric oxide, oxygen) to charge trace volatile organic compounds which are analysed in real time by a single quadrupole atmospheric ionisation (SIFT) mass spec. The charged multi-cluster VOC ions are known as product ions. In the techniques described in this paper, the mass spectrometry is switched to analyse both. Hence the precursor ions are adjusted and set to give optimum product ions of several mass clusters. By this method, the identification of a specific compound can only be made using a standard, and this standard must show the same multi-cluster product ions to allow assignment to be made.

Furthermore, Wagenstaller and Buettner, Metabolites 2013, 3, 637-657, describes the use of one- and two-dimensional high resolution gas chromatography-mass spectrometry (GC-MS) in combination with stable isotope dilution assays to quantify commonly occurring and potent odorants in human urine.

The use of mass spectrometric techniques to identify non-volatile cancer biomarkers in urine samples is also known in the art. Examples of such techniques, the cancer patients and the biomarkers identified therein are described in CN 104634907A (a combination of the amino acids alanine, glycine, valine, serine, threonine, proline, methionine and tyrosine in stomach cancer patients), WO 2013/162773 (betaine, malate, proline, N-acetylaspartate, uracil, xanthine, cysteine, alanine and N-acetyl glucosamine in prostate cancer patients), WO 2012/128508 (various non-volatile tags), WO 2010/104662 (various proteins), Soliman et al. J. Chromatography A, 2012, 1267, 162-169 (sarcosine in prostate cancer patients) and Struck et al. J. Chromatography A, 2013, 1283, 122-131 (the nucleosides 6-methyladenosine, inosine, N-2-methylguanosine, 3-methyluridine and N,N-dimethylguanosine, in urogenital cancer patients). However, none of these documents disclose that such biomarkers may be volatile and found in the headspace above the urine sample, or that such volatiles may be detected using mass spectrometry.

There remains a need in the art for improved techniques to more reliably determine the nature and levels of cancer biomarkers and thereby more easily identify patients with cancer using samples obtained from the patient.

### SUMMARY OF THE INVENTION

In one aspect, the invention provides a method of diagnosing cancer in a subject comprising determining a level of one or more volatile organic compounds in a urine sample obtained from the subject, wherein the identity and/or the level of the volatile organic compound is determined by mass spectrometry.

In one aspect, the invention provides a method of diagnosing cancer in a subject comprising determining a level of one or more volatile organic compounds in a urine sample obtained from the subject, wherein the identity and/or the level of the volatile organic compound is determined by mass spectrometry, and wherein the volatile organic compound is not formaldehyde.

In one aspect, the invention provides a method of diagnosing cancer in a subject comprising determining a level of one or more volatile organic compounds in a urine sample obtained from the subject, wherein the identity and/or the level of the volatile organic compound is determined by mass spectrometry, wherein the ionisation method in the mass spectrometry technique is electron impact ionisation.

In one aspect, the invention provides a method of diagnosing cancer in a subject comprising determining a level of one or more volatile organic compounds in a urine sample obtained from the subject, wherein the identity and/or the level of the volatile organic compound is determined by mass spectrometry, wherein the ionisation method in the mass spectrometry technique is proton ionisation.

In one aspect, the invention provides a method of diagnosing cancer in a subject comprising determining a level of one or more volatile organic compounds in a urine sample obtained from the subject, wherein the identity and/or the level of the volatile organic compound is determined by mass spectrometry, wherein the mass spectrometry technique is gas chromatography-mass spectrometry (GC-MS).

In one aspect, the invention provides a method of diagnosing cancer in a subject comprising determining a level of one or more volatile organic compounds in a urine sample obtained from the subject, wherein the identity and/or the level of the volatile organic compound is determined by mass spectrometry, wherein the mass spectrometry technique is tandem mass spectrometry.

In one aspect, the invention provides a method of diagnosing cancer in a subject comprising determining a level of one or more volatile organic compounds in a urine sample obtained from the subject, wherein the identity and/or the level of the volatile organic compound is determined by mass spectrometry, wherein the mass spectrometry technique is secondary electrospray ionisation (SESI) mass spectrometry.

In one aspect, the invention provides a method of diagnosing cancer in a subject comprising determining a level of one or more volatile organic compounds in a urine sample obtained from the subject, wherein the identity and/or the level of the volatile organic compound is determined by tandem mass spectrometry.

In one aspect, the invention provides a method of diagnosing cancer in a subject comprising determining a level of one or more volatile organic compounds in a urine sample obtained from the subject, wherein the identity and/or the level of the volatile organic compound is determined by mass spectrometry, and wherein the volatile organic compound does not have a mass-to-charge ratio (m/z) of 29 to 31 and a H⁺ pseudo molecular ion of m/z of 30 to 32.

Typically, the volatile organic compound is a biomarker for cancer. Therefore, in one aspect, the invention provides a method of diagnosing cancer in a subject comprising determining a level of one or more volatile biomarkers for cancer in a urine sample obtained from the subject, wherein the identity and/or the level of the biomarker is determined by mass spectrometry.

In one aspect, the invention provides a method of diagnosing cancer in a subject comprising determining a level of one or more volatile biomarkers for cancer in a urine sample obtained from the subject, wherein the identity and/or the level of the biomarker is determined by mass spectrometry, wherein the biomarker is not formaldehyde.

In one aspect, the invention provides a method of diagnosing cancer in a subject comprising determining a level of one or more volatile biomarkers for cancer in a urine sample obtained from the subject, wherein the identity and/or the level of the biomarker is determined by mass spectrometry, wherein the biomarker does not have a mass-to-charge ratio (m/z) of 29 to 31 and a H⁺ pseudo molecular ion of m/z of 30 to 32.

In one aspect, the invention provides a method of diagnosing cancer in a subject comprising determining a level of one or more volatile biomarkers for cancer in a urine sample obtained from the subject, wherein the identity and/or the level of the biomarker is determined by mass spectrometry, wherein the ionisation method in the mass spectrometry technique is electron impact ionisation.

In one aspect, the invention provides a method of diagnosing cancer in a subject comprising determining a level of one or more volatile biomarkers for cancer in a urine sample obtained from the subject, wherein the identity and/or the level of the biomarker is determined by mass spectrometry, wherein the ionisation method in the mass spectrometry technique is proton ionisation.

In one aspect, the invention provides a method of diagnosing cancer in a subject comprising determining a level of one or more volatile biomarkers for cancer in a urine sample obtained from the subject, wherein the identity and/or the level of the biomarker is determined by mass spectrometry, wherein the mass spectrometry technique is gas chromatography-mass spectrometry (GC-MS).

In one aspect, the invention provides a method of diagnosing cancer in a subject comprising determining a level of one or more volatile biomarkers for cancer in a urine sample obtained from the subject, wherein the identity and/or the level of the biomarker is determined by mass spectrometry, wherein the mass spectrometry technique is tandem mass spectrometry.

In one aspect, the invention provides a method of diagnosing cancer in a subject comprising determining a level of one or more volatile biomarkers for cancer in a urine sample obtained from the subject, wherein the identity and/or the level of the biomarker is determined by mass spectrometry, wherein the mass spectrometry technique is secondary electrospray ionisation (SESI) mass spectrometry.

In one aspect, the invention provides a method of diagnosing cancer in a subject comprising determining a level of one or more biomarkers for cancer in a urine sample obtained from the subject, wherein the level of the biomarker is determined by tandem mass spectrometry.

In another aspect, the invention provides a substance for use in a method of treating cancer in a subject, where the subject is identified as requiring treatment by a method as described above.

In another aspect, the invention provides a method of diagnosing cancer in a subject comprising determining a level of a biomarker for cancer in a urine sample obtained from the subject, wherein the biomarker has a mass-to-charge ratio (m/z) of 134 and a H⁺ pseudo molecular ion of m/z of 135.

In another aspect, the invention provides a method of diagnosing cancer in a subject comprising determining a level of a biomarker for cancer in a urine sample obtained from the subject, wherein the biomarker is an ethylbenzaldehyde or a dimethylbenzaldehyde.

In another aspect, the invention provides a method of diagnosing pancreatic cancer in a subject comprising determining a level of a biomarker for cancer in a urine sample obtained from the subject, wherein the biomarker has a mass-to-charge ratio (m/z) of 134 and a H⁺ pseudo molecular ion of m/z of 135.

In another aspect, the invention provides a method of diagnosing pancreatic cancer in a subject comprising determining a level of a biomarker for cancer in a urine sample obtained from the subject, wherein the biomarker is an ethylbenzaldehyde or a dimethylbenzaldehyde.

In another aspect, the invention provides a method of diagnosing lung cancer in a subject comprising determining a level of a biomarker for cancer in a urine sample obtained from the subject, wherein the biomarker has a mass-to-charge ratio (m/z) of 147 and a H⁺ pseudo molecular ion of m/z of 148.

In another aspect, the invention provides a method of diagnosing lung cancer in a subject comprising determining a level of a biomarker for cancer in a urine sample obtained from the subject, wherein the biomarker is 3-ethyl[1,2,4]triazolo[4,3-a]pyridine.

In another aspect, the invention provides a method of diagnosing lung cancer in a subject comprising determining a level of a biomarker for cancer in a urine sample obtained from the subject, wherein the biomarker has a mass-to-charge ratio (m/z) of 78 and a H⁺ pseudo molecular ion of m/z of 79.

In another aspect, the invention provides a method of diagnosing lung cancer in a subject comprising determining a level of a biomarker for cancer in a urine sample obtained from the subject, wherein the biomarker is benzene.

In another aspect, the invention provides a method of diagnosing breast cancer in a subject comprising determining a level of a biomarker for cancer in a urine sample obtained from the subject, wherein the biomarker has a mass-to-charge ratio (m/z) of 147 and a H⁺ pseudo molecular ion of m/z of 148.

In another aspect, the invention provides a method of diagnosing breast cancer in a subject comprising determining a level of a biomarker for cancer in a urine sample obtained from the subject, wherein the biomarker is 3-ethyl[1,2,4]triazolo[4,3-a]pyridine.

In another aspect, the invention provides a method of diagnosing prostate cancer in a subject comprising determining a level of a biomarker for cancer in a urine sample obtained from the subject, wherein the biomarker has a mass-to-charge ratio (m/z) of 186 and a H⁺ pseudo molecular ion of m/z of 187.

In another aspect, the invention provides a method of diagnosing prostate cancer in a subject comprising determining a level of a biomarker for cancer in a urine sample obtained from the subject, wherein the biomarker is 1-dodecanol.

In another aspect, the invention provides a method of diagnosing renal cancer in a subject comprising determining a level of a biomarker for cancer in a urine sample obtained from the subject, wherein the biomarker has a mass-to-charge ratio (m/z) of 147 and a H⁺ pseudo molecular ion of m/z of 148.

In another aspect, the invention provides a method of diagnosing renal cancer in a subject comprising determining a level of a biomarker for cancer in a urine sample obtained from the subject, wherein the biomarker is menthol.

In another aspect, the invention provides a method of diagnosing renal cancer in a subject comprising determining a level of a biomarker for cancer in a urine sample obtained from the subject, wherein the biomarker has a mass-to-charge ratio (m/z) of 130 and a H⁺ pseudo molecular ion of m/z of 131.

In another aspect, the invention provides a method of diagnosing renal cancer in a subject comprising determining a level of a biomarker for cancer in a urine sample obtained from the subject, wherein the biomarker is 2-ethyl-1-hexanol.

In another aspect, the invention provides a method of diagnosing bladder cancer in a subject comprising determining a level of a biomarker for cancer in a urine sample obtained from the subject, wherein the biomarker has a mass-to-charge ratio (m/z) of 156 and a H⁺ pseudo molecular ion of m/z of 157.

In another aspect, the invention provides a method of diagnosing bladder cancer in a subject comprising determining a level of a biomarker for cancer in a urine sample obtained from the subject, wherein the biomarker is menthol.

In another aspect, the invention provides a method of diagnosing colorectal cancer in a subject comprising determining a level of a biomarker for cancer in a urine sample obtained from the subject, wherein the biomarker has a mass-to-charge ratio (m/z) of 94 and a H⁺ pseudo molecular ion of m/z of 95.

In another aspect, the invention provides a method of diagnosing bladder cancer in a subject comprising determining a level of a biomarker for cancer in a urine sample obtained from the subject, wherein the biomarker is phenol.

In another aspect, the invention provides a method of diagnosing colorectal cancer in a subject comprising determining a level of a biomarker for cancer in a urine sample obtained from the subject, wherein the biomarker has a mass-to-charge ratio (m/z) of 98 and a H⁺ pseudo molecular ion of m/z of 99.

In another aspect, the invention provides a method of diagnosing bladder cancer in a subject comprising determining a level of a biomarker for cancer in a urine sample obtained from the subject, wherein the biomarker is cyclohexanone.

In another aspect, the invention provides a method of diagnosing colorectal cancer in a subject comprising determining a level of a biomarker for cancer in a urine sample obtained from the subject, wherein the biomarker has a mass-to-charge ratio (m/z) of 130 and a H⁺ pseudo molecular ion of m/z of 131.

In another aspect, the invention provides a method of diagnosing bladder cancer in a subject comprising determining a level of a biomarker for cancer in a urine sample obtained from the subject, wherein the biomarker is 2-ethyl-1-hexanol.

In another aspect, the invention provides a method of diagnosing colorectal cancer in a subject comprising determining a level of a biomarker for cancer in a urine sample obtained from the subject, wherein the biomarker has a mass-to-charge ratio (m/z) of 116 and a H⁺ pseudo molecular ion of m/z of 117.

In another aspect, the invention provides a method of diagnosing bladder cancer in a subject comprising determining a level of a biomarker for cancer in a urine sample obtained from the subject, wherein the biomarker is butyl acetate.

In another aspect, the invention provides an ethylbenzaldehyde or a dimethylbenzaldehyde for use in a method of treating cancer in a subject, where the subject is identified as requiring treatment by a method as defined above.

These and further aspects of the invention are described herein.

### DETAILED DESCRIPTION

Urine Sample

The methods of the invention involve analysis of urine samples obtained from a subject. As is clear to the person skilled in the art, obtaining a urine sample simply requires the subject to urinate and does not require any surgical or invasive procedure to be carried out on the patient.

In one embodiment, the method of the invention is carried out by analysing, using mass spectrometry, volatile organic compounds (as defined below) in a urine sample obtained from the subject. Typically, such volatile compounds are present in the headspace above the urine sample. As is known to the person skilled in the art, the headspace comprises the air surrounding the urine sample, into which the volatile organic compounds evaporate and/or diffuse. Typically, the headspace comprises, consists essentially of, or consists of, the air in a closed container containing the urine sample.

Volatile Organic Compounds

The method of the invention comprises the analysis by a mass spectrometric technique as defined and exemplified herein of volatile organic compounds in a urine sample obtained from the subject.

The term “volatile organic compound” is defined in various different ways in the art. The US Environmental Protection Agency (EPA) defines a volatile organic compound (VOC) with reference to its role in air pollution and therefore defines a VOC as meaning any compound of carbon, excluding carbon monoxide, carbon dioxide, carbonic acid, metallic carbides or carbonates and ammonium carbonate, which participates in atmospheric photochemical reactions, except those designated by EPA as having negligible photochemical reactivity. According to this definition, VOCs are organic chemical compounds whose composition makes it possible for them to evaporate under normal indoor atmospheric conditions of temperature and pressure. This is the general definition of VOCs that is used in the scientific literature, and is consistent with the definition used for indoor air quality. Since the volatility of a compound is generally higher the lower its boiling point temperature, the volatility of organic compounds are sometimes defined and classified by their boiling points.

The European Union uses the boiling point of the compound, rather than its volatility, in its definition of VOCs. According to the EU definition, a VOC is any organic compound having an initial boiling point less than or equal to 250° C. measured at a standard atmospheric pressure of 101.3 kPa.

Alternatively, VOCs can be categorized by the ease they will be emitted. For example, the World Health Organization (VVHO) divides indoor organic pollutants into the following categories:
Very volatile organic compounds (WOCs)
Volatile organic compounds (VOCs)
Semi-volatile organic compounds (SVOCs)

The higher the volatility (lower the boiling point), the more likely the compound will be emitted from a product or surface into the air. Very volatile organic compounds are so volatile that they are difficult to measure and are found almost entirely as gases in the air rather than in materials or on surfaces. The least volatile compounds found in air constitute a far smaller fraction of the total present indoors while the majority will be in solids or liquids that contain them or on surfaces including dust, furnishings and building materials. Based on the above WHO definition, the three classes of volatiles can be classed as follows:
A VVOC can be defined as having a minimum boiling point from less than or equal to 0° C. and a maximum boiling point of 50 to 100° C.
A VOC can be defined as having a minimum boiling point from 50 to 100° C. and a maximum boiling point of 240 to 260° C.
A SVOC can be defined as having a minimum boiling point from 240 to 260° C. and a maximum boiling point of 380 to 400° C.

In one embodiment, the volatile organic compound is a WOC, VOC or SVOC according to any one of the above WHO definitions. In one embodiment, the volatile organic compound is a WOC or VOC according to any one of the above WHO definitions. In one embodiment, the volatile organic compound is a VOC according to any one of the above WHO definitions.

In one embodiment, the volatile organic compound exerts a vapour pressure at room temperature (defined herein as 20 to 30° C., preferably 23 to 27° C., more preferably 24 to 26° C. and most preferably 25° C.). In one embodiment, the volatile organic compound exerts a vapour pressure at a temperature above room temperature (defined herein as 30 to 80° C., preferably 40 to 70° C., more preferably 45 to 60° C. and most preferably 50° C.).

In one embodiment, the volatile organic compound has a headspace concentration from 1 part per quadrillion to 100 parts per thousand (1%). In one embodiment, the volatile organic compound has a headspace concentration from 0.001 part per trillion to 10 parts per thousand (0.1%). In one embodiment, the volatile organic compound has a headspace concentration from 0.01 part per trillion to 1 part per thousand (0.001%). In one embodiment, the volatile organic compound has a headspace concentration from 0.1 part per trillion to 100 parts per million. In one embodiment, the volatile organic compound has a headspace concentration from 1 parts per trillion to 10 parts per million. In one embodiment, the volatile organic compound has a headspace concentration from 10 parts per trillion to 1 part per million. In one embodiment, the volatile organic compound has a headspace concentration from 100 parts per trillion to 100 parts per billion. In this specification, the above parts per number (thousand, million, billion, trillion, quadrillion) of headspace concentrations expressed above are by mass of the total mass of air in the headspace above the urine sample. The headspace concentrations may be measured, for example, using the methods described generally in Gujral et al. Am. J. Sci. Ind. Res. 2010, 1(2), 271-278. Typically, the headspace concentration is measured at a temperature between room temperature (as defined above) and 80° C., preferably 40 to 60° C., and more preferably 50° C.

In one embodiment, the volatile organic compound has a boiling point of at most 280° C. In one embodiment, the volatile organic compound has a boiling point of at most 270° C. In one embodiment, the volatile organic compound has a boiling point of at most 260° C. In one embodiment, the volatile organic compound has a boiling point of at most 250° C. In one embodiment, the volatile organic compound has a boiling point of at most 240° C. In one embodiment, the volatile organic compound has a boiling point of at most 230° C. In one embodiment, the volatile organic compound has a boiling point of at most 220° C. In one embodiment, the volatile organic compound has a boiling point of at most 210° C. In one embodiment, the volatile organic compound has a boiling point of at most 200° C. In one embodiment, the volatile organic compound has a boiling point of at most 190° C. In one embodiment, the volatile organic compound has a boiling point of at most 180° C. In one embodiment, the volatile organic compound has a boiling point of at most 170° C. In one embodiment, the volatile organic compound has a boiling point of at most 160° C. In one embodiment, the volatile organic compound has a boiling point of at most 150° C. In one embodiment, the volatile organic compound has a boiling point of at most 140° C. In one embodiment, the volatile organic compound has a boiling point of at most 130° C. In one embodiment, the volatile organic compound has a boiling point of at most 120° C. In one embodiment, the volatile organic compound has a boiling point of at most 110° C. In one embodiment, the volatile organic compound has a boiling point of at most 100° C. In one embodiment, the volatile organic compound has a boiling point of at most 90° C. In one embodiment, the volatile organic compound has a boiling point of at most 80° C. In one embodiment, the volatile organic compound has a boiling point of at most 70° C. In one embodiment, the volatile organic compound has a boiling point of at most 60° C. In one embodiment, the volatile organic compound has a boiling point of at most 50° C. In one embodiment, the volatile organic compound has a boiling point of at most 40° C. In one embodiment, the volatile organic compound has a boiling point of at most 30° C. All of the above boiling points are expressed at normal atmospheric pressure (101.3 kPa).

In one embodiment, the volatile organic compound has a boiling point of between room temperature (as defined above) and 280° C. In one embodiment, the volatile organic compound has a boiling point of between room temperature and 270° C. In one embodiment, the volatile organic compound has a boiling point of between room temperature and 260° C. In one embodiment, the volatile organic compound has a boiling point of between room temperature and 250° C. In one embodiment, the volatile organic compound has a boiling point of between room temperature and 240° C. In one embodiment, the volatile organic compound has a boiling point of between room temperature and 230° C. In one embodiment, the volatile organic compound has a boiling point of between room temperature and 220° C. In one embodiment, the volatile organic compound has a boiling point of between room temperature and 210° C. In one embodiment, the volatile organic compound has a boiling point of between room temperature and 200° C. In one embodiment, the volatile organic compound has a boiling point of between room temperature and 190° C. In one embodiment, the volatile organic compound has a boiling point of between room temperature and 180° C. In one embodiment, the volatile organic compound has a boiling point of between room temperature and 170° C. All of the above boiling points are expressed at normal atmospheric pressure (101.3 kPa).

The minimum boiling point of the volatile organic compound may range from room temperature upwards, for example 30° C., 40° C., 50° C., 60° C., 70° C., 80° C., 90° C., 100° C., 110° C., 120° C., 130° C., 140° C., 150° C., 160° C., 170° C., 180° C., 190° C., 200° C., 210° C., 220° C., 230° C., 240° C., 250° C., 260° C., 270° C. or 280° C. Typically the boiling point of the volatile organic compound may range from any of the above minima to any of the maxima set out above. All of the above boiling points are expressed at normal atmospheric pressure (101.3 kPa).

In one embodiment, the volatile organic compound has a boiling point of between room temperature and 160° C. In one embodiment, the volatile organic compound has a boiling point of between room temperature and 150° C. In one embodiment, the volatile organic compound has a boiling point of between room temperature and 140° C. In one embodiment, the volatile organic compound has a boiling point of between room temperature and 130° C. In one embodiment, the volatile organic compound has a boiling point of between room temperature and 120° C. In one embodiment, the volatile organic compound has a boiling point of between room temperature and 110° C. In one embodiment, the volatile organic compound has a boiling point of between room temperature and 100° C. In one embodiment, the volatile organic compound has a boiling point of between room temperature and 90° C. In one embodiment, the volatile organic compound has a boiling point of between room temperature and 80° C. In one embodiment, the volatile organic compound has a boiling point of between room temperature and 70° C. In one embodiment, the volatile organic compound has a boiling point of between room temperature and 60° C. In one embodiment, the volatile organic compound has a boiling point of between room temperature and 50° C. In one embodiment, the volatile organic compound has a boiling point of between room temperature and 40° C. In one embodiment, the volatile organic compound has a boiling point of between room temperature and 30° C. All of the above boiling points are expressed at normal atmospheric pressure (101.3 kPa).

The volatile organic compound may also be defined by its molecular mass. The mass is measured in unified atomic mass units (u). As is clear to the person skilled in the art, the molecular mass is the same as the mass-to-charge ratio (m/z) for singly charged mass ions (i.e. when z=1). In this specification all molecular mass and mass-to-charge ratios are expressed to the nearest whole number. The mass of the volatile organic compound is therefore the same as the m/z of the mass ion (M⁺ or M) when the mass spectrometry involves electron impact ionisation. When the mass spectrometry involves atmospheric soft ionisation such as electrospray, secondary electrospray ionisation, or atmospheric chemical ionisation, the mass of chemical species (e.g. H⁺ or Na⁺) needs to be deducted from the mass of the species identified to give the mass of the molecular ion identified. The mass ion formed by such soft ionisation techniques is termed the “pseudo molecular ion”. In one embodiment the pseudo molecular ion is [M+H]⁺. In one embodiment the pseudo molecular ion is [M+Na]⁺. In one embodiment the pseudo molecular ion is [M+NH₄]⁺. In one embodiment the pseudo molecular ion is [M+H]⁻.

In one embodiment, the volatile organic compound has a molecular mass of 16 to 290 u. In one embodiment, the volatile organic compound has a molecular mass of 32 to 290 u. In one embodiment, the volatile organic compound has a molecular mass of 32 to 250 u. In one embodiment, the volatile organic compound has a molecular mass of 32 to 210 u. In one embodiment, the volatile organic compound has a molecular mass of 32 to 206 u. In one embodiment, the volatile organic compound has a molecular mass of 50 to 200 u. In one embodiment, the volatile organic compound has a molecular mass of 70 to 180 u. In one embodiment, the volatile organic compound has a molecular mass of 90 to 160 u.

In one embodiment, the volatile organic compound has a molecular mass of 32 to 40 u. In one embodiment, the volatile organic compound has a molecular mass of 40 to 50 u. In one embodiment, the volatile organic compound has a molecular mass of 50 to 60 u. In one embodiment, the volatile organic compound has a molecular mass of 60 to 70 u. In one embodiment, the volatile organic compound has a molecular mass of 70 to 80 u. In one embodiment, the volatile organic compound has a molecular mass of 80 to 90 u. In one embodiment, the volatile organic compound has a molecular mass of 90 to 100 u. In one embodiment, the volatile organic compound has a molecular mass of 100 to 110 u. In one embodiment, the volatile organic compound has a molecular mass of 110 to 120 u. In one embodiment, the volatile organic compound has a molecular mass of 120 to 130 u. In one embodiment, the volatile organic compound has a molecular mass of 130 to 140 u. In one embodiment, the volatile organic compound has a molecular mass of 140 to 150 u. In one embodiment, the volatile organic compound has a molecular mass of 150 to 160 u. In one embodiment, the volatile organic compound has a molecular mass of 160 to 170 u. In one embodiment, the volatile organic compound has a molecular mass of 170 to 180 u. In one embodiment, the volatile organic compound has a molecular mass of 180 to 190 u. In one embodiment, the volatile organic compound has a molecular mass of 190 to 200 u. In one embodiment, the volatile organic compound has a molecular mass of 200 to 210 u.

In one embodiment, the volatile organic compound has a molecular mass of 134 u. In one embodiment, the volatile organic compound has a molecular mass of 186 u. In one embodiment, the volatile organic compound has a molecular mass of 206 u. In one embodiment, the volatile organic compound has a molecular mass of 156 u. In one embodiment, the volatile organic compound has a molecular mass of 81 u. In one embodiment, the volatile organic compound has a molecular mass of 128 u. In one embodiment, the volatile organic compound has a molecular mass of 114 u. In one embodiment, the volatile organic compound has a molecular mass of 142 u. In one embodiment, the volatile organic compound has a molecular mass of 156 u. In one embodiment, the volatile organic compound has a molecular mass of 128 u. In one embodiment, the volatile organic compound has a molecular mass of 114 u. In one embodiment, the volatile organic compound has a molecular mass of 147 u. In one embodiment, the volatile organic compound has a molecular mass of 150 u. In one embodiment, the volatile organic compound has a molecular mass of 86 u. In one embodiment, the volatile organic compound has a molecular mass of 108 u. In one embodiment, the volatile organic compound has a molecular mass of 45 u. In one embodiment, the volatile organic compound has a molecular mass of 112 u. In one embodiment, the volatile organic compound has a molecular mass of 152 u. In one embodiment, the volatile organic compound has a molecular mass of 196 u. In one embodiment, the volatile organic compound has a molecular mass of 46 u. In one embodiment, the volatile organic compound has a molecular mass of 58 u. In one embodiment, the volatile organic compound has a molecular mass of 110 u. In one embodiment, the volatile organic compound has a molecular mass of 32 u. In one embodiment, the volatile organic compound has a molecular mass of 152 u. In one embodiment, the volatile organic compound has a molecular mass of 98 u. In one embodiment, the volatile organic compound has a molecular mass of 94 u. In one embodiment, the volatile organic compound has a molecular mass of 120 u. In one embodiment, the volatile organic compound has a molecular mass of 116 u.

In one embodiment, the volatile organic compound does not have a molecular mass of 29 to 31 u. In one embodiment, the volatile organic compound does not have a molecular mass of 30 u.

General Method Steps

The method of the invention is carried out by analysing, using mass spectrometry, volatile organic compounds in a urine sample obtained from the subject. In some embodiments, the sample is treated prior to the mass spectrometry analysis being carried out.

In one embodiment, the urine sample is treated with a base (typically an alkali, i.e. a water-soluble base) prior to the mass spectrometry. Unexpectedly, it has been found that treatment with alkali releases a greater proportion of the volatile organic compounds from the liquid urine into the headspace above the urine, thereby increasing the sensitivity and hence the specificity of the mass spectrometry analysis. Examples of suitable alkalis are well known to the person skilled in the art, and include alkali metal hydroxides such as sodium hydroxide and potassium hydroxide, alkaline earth metal hydroxides such as calcium hydroxide, and ammonium hydroxide. Treatment with alkali is particularly useful when the mass spectrometry is gas chromatography-mass spectrometry (GCMS).

Typically, the urine sample which undergoes mass spectrometry analysis is treated with alkali such that the pH is between 9 and 14, preferably between 11 and 14, and most preferably between 13 and 14.

In one embodiment, the urine sample headspace is exposed to an absorbent or absorbents prior to the mass spectrometry analysis. It has been found that treatment with absorbent or absorbents increases the concentration of volatile organic compounds in the headspace above the urine thereby increasing the efficiency and the accuracy of the mass spectrometry. Examples of absorbent compounds include poly(2,6-diphenylphenylene oxide) (Tenax®, such as (Tenax TA (mesh 20-35, 35-60, 60-80, 80-100, 100-200) and graphitised poly(2,6-diphenylphenylene oxide) (Tenax GR), such as 23% graphite (mesh 20-35, 35-60, 60-80, 80-100) (all available from Buchem B.V.); polydimethylsiloxane (PDMS), carbon molecular sieve (e.g. Carboxen® such as Carboxen® (1000 (80-100 mesh), 1003 (40-60 mesh), or 569 (20-40 mesh), or Carbosieve SIII (available from Sigma-Aldrich); divinylbenzene; methoxypolyethylene glycols (MPEGs) (Carbowax®, available from Dow Chemical), spherical porous polymer beads such as Porapak® (Q and N), or Hayesep D (all available from Sigma-Aldrich), Carbograph (1TD, 2TD, 4TD, 5TD) (available from Alltech Associates Inc.), used alone or as a mixture of any of these.

In the above embodiment where the urine headspace is exposed to an absorbent or absorbents, a desorption is preferably carried out on a urine sample prior to the mass spectrometry. It has been found that desorption releases the volatile organic compounds from the absorbent, thereby increasing the sensitivity and the selectivity of the mass spectrometry analysis. When the mass spectrometry is gas chromatography-mass spectrometry (GCMS), the desorption is typically carried out by thermal desorption, with the heater typically heated to between 180° C. and 280° C. When the mass spectrometry is secondary electrospray-mass spectrometry (SESI MS), the desorption is typically carried out by the electrospray plume.

Mass Spectrometry

The present invention relates generally to the use of mass spectrometry techniques to identify cancer biomarkers in a urine sample obtained from a subject.

As is known to the person skilled in the art, a mass spectrometer typically consists of three components: an ion source, a mass analyzer, and a detector. The ionizer converts a portion of the sample into ions. As detailed below, there are a wide variety of ionization techniques, depending on the phase (solid, liquid, gas) of the sample and the efficiency of various ionization mechanisms for the unknown species. The mass spectrometer also typically comprises an extraction system which removes ions from the sample, which are then targeted through the mass analyzer and onto the detector. The difference in mass-to-charge (m/z) of the fragments allows the mass analyzer to sort the ions by their mass-to-charge ratio. Finally, the detector measures the value of an indicator quantity and thus provides data for calculating the abundances of each ion present.

In a typical mass spectrometry procedure, the first step comprises ionization of a sample. In one embodiment, the ionization comprises electron ionization (El), which comprises bombarding the sample with electrons. In another embodiment, the ionization comprises chemical ionization (CI), according to which ions are produced through the collision of the analyte with ions of a reagent gas that are present in the ion source (examples of suitable reagent gases include methane, ammonia, and isobutane). In another embodiment, the ionization comprises Atmospheric Pressure Chemical Ionisation (APCI). In another embodiment, the ionization comprises Atmospheric Pressure Photon Ionization (APPI).

When the ionization is electron ionization, this typically results in a mass ion having the same mass (M) as the parent molecule but a charge (M⁺ or M⁻). When the ionization is chemical ionization, this typically results in a mass ion having a mass of the parent molecule and the chemical species used to ionize the molecule, well known examples including [M+H]⁺, [M−H]⁻, [M+NH₄]⁺ and [M+Na]⁺. Such a molecular ion is also referred to in this specification as a “pseudo molecular ion”

In another embodiment, the ionization comprises electrospray ionization (ESI), in which the liquid containing the analyte(s) of interest is dispersed by electrospray into a fine aerosol. In another embodiment, the ionization comprises matrix-assisted laser desorption/ionization (MALDI), which typically comprises a three-step process, as follows: (1) mixing the sample is a suitable matrix material and applying it to a surface, typically a metal plate; (2) irradiating the sample, typically with a pulsed laser, thereby triggering ablation and desorption of the sample and matrix material; and (3) ionization of the analyte molecules by being protonated or deprotonated in the hot plume of ablated gases, allowing the ions to be accelerated into the mass spectrometer used to analyse them. These ionization techniques are well known to the person skilled in the art. Ionization, in particular electron ionization, may cause some of the sample's molecules to break into charged fragments.

Following ionization, the ions produced in the first step are then separated according to their mass-to-charge (m/z) ratio in the mass analyzer. This is typically carried out by one or more of the following mass to charge separation techniques: by quadrupole electric fields as used in quadrupole mass spectrometers, by ion trap quadrupole electric fields as used by ion trap mass spectrometers, by longitudinal ion travelling time as used by time of flight mass spectrometers and by electric and/or magnetic field deflection as traditionally used by electric and magnetic sector mass spectrometers. This last technique involves accelerating the ions and subjecting them to an electric or magnetic field, such that the electric or magnetic field causes the ions to be deflected. Ions of the same mass-to-charge ratio will undergo the same amount of deflection.

Following separation, the ions are detected. Typically, the detector records either the charge induced or the current produced when an ion passes by or hits a surface. In a scanning instrument, the signal produced in the detector during the course of the scan versus where the instrument is in the scan will produce a mass spectrum, a record of ions as a function of m/z.

In one embodiment, mass spectrometry is used in tandem with a chromatographic separation technique. In one embodiment, the chromatographic technique is gas chromatography, the combination technique being known as gas chromatography-mass spectrometry (GC/MS, GCMS or GC-MS). As is known to the person skilled in the art, in this technique, a gas chromatograph is used to separate different compounds. This stream of separated compounds is fed into the mass spectrometer for ionization, mass analysis and detection as described above.

When the mass spectrometry is gas chromatography-mass spectrometry (GCMS) it is especially preferred that the urine sample is treated with a base (typically an alkali, as defined and exemplified above) Unexpectedly, it has been found that treatment with alkali releases a greater proportion of volatile organic compounds from the liquid urine into the headspace above the urine, and a greater portion are absorbed on to the absorbent thereby increasing the sensitivity and the specificity of the mass spectrometry analysis.

In one embodiment, the chromatographic technique is liquid chromatography, the combination technique being known as liquid chromatography-mass spectrometry (LC/MS, LCMS or LC-MS). As is known to the person skilled in the art, this technique separates compounds chromatographically using a liquid mobile phase. Typically, the liquid phase is a mixture of water and organic solvents. The stream of separated compounds is then fed into the mass spectrometer for ionization, mass analysis and detection as described above.

In one embodiment, the mass spectrometry is carried out without a prior chromatographic step. In this embodiment, the sample can be introduced directly into the mass spectrometer.

In one embodiment, the mass spectrometry is direct sampling mass spectrometry. As is known to the person skilled in the art, this technique involves the introduction of a sampling probe containing the sample to be analysed directly into the ionisation chamber of the mass spectrometer. The sample may be solid, liquid or gas, preferably solid.

In one embodiment, the mass spectrometry is infusion sampling mass spectrometry. As is known to the person skilled in the art, this technique involves the introduction of the sample to be analysed into the mass spectrometer by spraying a liquid containing the sample into the mass spectrometer.

In one embodiment, the level of the biomarker is determined by tandem mass spectrometry. Tandem mass spectrometry, also known as MS/MS, MS² or MSⁿ (where n is at least 2, preferably 2 to 10, more preferably 2 to 5, even more preferably 2 or 3, most preferably 2) involves multiple steps of mass spectrometry selection, with some form of fragmentation occurring in between the steps. Typically, tandem mass spectrometry and similar techniques allow analysis to be carried out on the daughter ions resulting from the fragmentation as well as the parent ion, the fragmentation pattern allowing improved identification of the volatile organic compounds and differentiation of those compounds where the parent ion has the same molecular mass.

In the present invention, tandem mass spectrometry entails specific advantages over other mass spectrometric techniques, and particularly over the single quadrupole and SIFT (& SYFT) MS techniques used in the prior art to analyse volatile organic compounds in urine samples, in that greater specificity (and sensitivity) can be routinely obtained. The specificity attained by tandem mass spectrometry in selecting a single mass ion from a complex matrix of analytes can avoid the use of a full chromatographic separation step prior to mass spectrometry analysis. This approach can afford a rapid screening and/or a high throughput of samples as it allows a chromatographic separation step to be omitted. Tandem mass spectrometry therefore confers the potential to differentiate between cancerous and non-cancerous urine samples by determining the identity and abundance of biomarker mass ion(s) in a more rapid, more specific and/or more sensitive manner than was previously possible in the art.

Typically, tandem mass spectrometry involves the following steps:

(a) Ionization of a sample to produce ions. The ionization may be carried out using any of the ionization techniques generally described above, in particular Electron Impact (El), Electrospray Ionization (ESI), Secondary Electrospray Ionization (SESI), Desorption Electrospray Ionization (DESI), Easy Ambient Sonic Spray Ionisation (EASI), Extractive Electrospray Ionization (FESI), Neutral Desorption Electrospray Ionization (ND-ESI), Jet Desorption Electrospray Ionization (JEDI), Liquid Extraction Surface Analysis (LESA), Surface Activated Chemical Ionization (SACI), Atmospheric Pressure Chemical Ionization (APCI), Atmospheric Pressure Photon Ionization (APPI), Direct Analysis in Real Time (DART), or Matrix Assisted Laser Desorption Ionization (MALDI).

(b) Separating the ions according to their mass-to-charge ratio to produce one or more precursor ions. The separation is carried out as generally described above.

(c) Fragmentation of the one or more separated precursor ions to yield a product ion. There are many methods used to fragment the ions and these can result in different types of fragmentation and thus different information about the structure and composition of the molecule. In one embodiment, the fragmentation method comprises collision-induced dissociation. Typically, this method involves the collision of an ion with a neutral atom or molecule in the gas phase and subsequent dissociation of the ion. In one embodiment, the fragmentation method comprises an electron impact capture and/or transfer method. Typically, this method uses the energy released when an electron is transferred to or captured by a multiply charged ion to induce fragmentation. Examples of electron capture and/or transfer methods used to induce fragmentation include electron capture dissociation, electron transfer dissociation, negative electron transfer dissociation, electron-detachment dissociation, and charge transfer dissociation. In one embodiment, the fragmentation method comprises photodissociation. Typically, in this method the energy required for dissociation can be added by photon absorption. Examples of photodissociation methods include infrared multi-photon dissociation, blackbody infrared radiative dissociation or surface induced dissociation. In another embodiment, the fragmentation technique comprises in-source fragmentation (i.e. fragmentation in the ionization chamber) in which the ionization process is sufficiently violent to leave the resulting ions with sufficient internal energy to fragment within the mass spectrometer (e.g. by electron impact, Chemical Ionization or “accelerated ion dissociation”). All of these techniques are well known to the person skilled in the art.

(d) Separating the product ions obtained from the fragmentation process according to their mass-to-charge ratio. The separation is typically carried out as generally described above.

(e) Detection of the separated ions. The detection is typically carried out as generally described above.

In one embodiment, the tandem mass spectrometry is ion trap mass spectrometry. As is known to the person skilled in the art, a quadrupole ion trap is a type of ion trap that uses dynamic electric fields to trap charged particles.

In one embodiment, the tandem mass spectrometry is triple quadrupole mass spectrometry (TAMS). As is known to the person skilled in the art, a triple quadrupole mass spectrometer is a tandem mass spectrometer consisting of two quadrupole mass analyzers in series, with a (non-mass-resolving) radio frequency-only quadrupole between them to act as a cell for collision-induced dissociation.

In one embodiment, the tandem mass spectrometry is quadrupole time of flight mass spectrometry. As is known to the person skilled in the art, a quadrupole time-of-flight mass spectrometer is a triple quadrupole mass spectrometer, as described above, with the final quadrupole replaced by a time-of-flight device. As is known to the person skilled in the art, time-of-flight mass spectrometry (TOFMS) is a method of mass spectrometry in which the mass-to-charge ratio (m/z) of an ion is determined via a time measurement. The technique involves acceleration of the ions by an electric field of known strength. This acceleration results in an ion having the same kinetic energy as any other ion that has the same charge. The velocity of the ion depends on the mass-to-charge ratio. The time that it subsequently takes for the particle to reach a detector at a known distance is measured. This time will depend on the mass-to-charge ratio of the particle, heavier particles reaching lower speeds. From this time and the known experimental parameters the user can determine the mass-to-charge ratio of the ion.

In one embodiment, the tandem mass spectrometry is Quadrupole Ion Trap mass spectrometry. In one embodiment, the tandem mass spectrometry is Quadrupole-Time of Flight mass spectrometry. In one embodiment, the tandem mass spectrometry is Ion Mobility-Quadrupole Ion Trap-Time of Flight mass spectrometry. In one embodiment, the tandem mass spectrometry is Quadrupole-Orbitrap mass spectrometry. In one embodiment, the tandem mass spectrometry is Quadrupole Ion Trap mass spectrometry. In one embodiment, the tandem mass spectrometry is Ion Mobility Spectrometer-Quadrupole ion Trap mass spectrometry. In one embodiment, the tandem mass spectrometry is Quadrupole-Orbitrap Mass spectrometry. In one embodiment, the tandem mass spectrometry is a Triple-Quadrupole-Orbitrab mass spectrometry. In one embodiment, the tandem mass spectrometry is Quadrupole Ion Trap-Orbitrap mass spectrometry. In one embodiment, the tandem mass spectrometry is Time of Flight, Ion Trap-Fourier Transform mass spectrometry. Details of these techniques are known to the person skilled in the art.

In one embodiment, the tandem mass spectrometry is secondary electrospray ionization (SESI) mass spectrometry. SESI is an electrospray ionzation technique carried out at atmospheric pressure. The term “SESI” generally covers a range of modified ESI techniques where the electrospray ionization plume ionises VOC's in the immediate vicinity of the electrospray plume. Typically, a SESI technique is carried out within a ionisation chamber in front of the skimmer entrance of an atmospheric pressure ionization mass spectrometer. In one embodiment, the urine samples is heated to between room temperature (25° C.) to 200° C., preferably between 50° C. and 150° C., more preferably between 80° C. to 120° C. In one embodiment, the urine sample is in close proximity (typically 0-10 mm) to the electrospray plume. In this technique the urine sample is preferably treated with alkali (as defined and exemplified above) prior to introduction into the ionization chamber. In one embodiment, the sample introduced is an absorbent, described and exemplified above in which have been collected or absorbed the headspace VOC compounds released from the urine samples (preferably alkali treated urine samples, described and exemplified above) In this embodiment, the absorbent may be within the electrospray plume. In one embodiment, the sample is a VOC absorbent, described and exemplified above in which have been collected or absorbed the headspace VOC compounds released from urine samples (preferably alkali treated urine samples), described and exemplified above, were heated between room temperature (25° C. to 250° C.), preferably between 50° C. and 220° C., more preferably between 150° C. to 200° C. Typically, the absorbent is in close proximity (typically 0-10 mm) of the electrospray plume. This technique is described in more detail in Bean, H. D., Zhu, J., Hill, J. E., “Characterizing Bacterial Volatiles using Secondary Electrospray Ionization Mass Spectrometry (SESI-MS)”. J. Vis. Exp. (52), e2664, doi:10.3791/2664 (2011).

When the mass spectrometry is SESI mass spectrometry, preferably the urine sample is treated with an absorbent (as defined and exemplified above) prior to introduction into the SESI mass spectrometer. Unexpectedly, it has been found that treatment with absorbent increases the concentration of volatile organic compounds in the headspace above the urine thereby increasing the efficiency and the accuracy of the mass spectrometry analysis.

Typically, the heated urine sample is placed close to the entrance (for example, up to 50 mm, preferably up to 5 mm) of the mass spectrometer. When the mass spectrometer has a skimmer cone entrance, the heated urine sample is typically placed close to the skimmer cone entrance (for example, up to 30 mm, preferably up to 5 mm) of the mass spectrometer. The electrospray plume ionizes the plume of released volatilised VOC compounds. Typically, the absorbent is placed close to the entrance (for example, up to 50 mm, preferably up to 5 mm) of the mass spectrometer. When the mass spectrometer has a skimmer cone entrance, the absorbent is typically placed close to the skimmer cone entrance (for example, up to 30 mm, preferably up to 5 mm) of the mass spectrometer. The electrospray plume impedes onto the absorbent releasing and ionizes the absorbed VOC compounds.

Typically, the heated absorbent is placed close to the entrance (for example, up to 50 mm, preferably up to 5 mm) of the mass spectrometer. When the mass spectrometer has a skimmer cone entrance, the absorbent is typically placed close to the skimmer cone entrance (for example, up to 30 mm, preferably up to 5 mm) of the mass spectrometer. The electrospray plume ionizes the plume of released volatilised VOC compounds.

When the mass spectrometry is SESI mass spectrometry, a desorption is preferably carried out on the absorbed volatiles. The desorption may be carried out by the electrospray plume by thermal desorption as defined and exemplified above, or a combination thereof.

Diagnosis of Cancer Using Biomarkers Identified by Mass Spectrometry

Following separation and analysis by the appropriate mass spectrometric method, the volatile organic compounds identified in the subject's urine sample can be used to detect cancer in the subject. Typically, this step comprises comparing the level of the one or more volatile organic compounds in the urine sample to a reference value, wherein the level of the one or more volatile organic compounds in the urine sample compared to the reference value is indicative of cancer in the subject.

In one embodiment, a decrease in the level of the one or more volatile organic compounds in the urine sample compared to the reference value is indicative of cancer in the subject. In one embodiment, an increase in the level of the one or more volatile organic compounds in the urine sample compared to the reference value is indicative of cancer in the subject.

In one embodiment, the volatile organic compound (as defined and exemplified above) is a biomarker for cancer and the step comprises comparing the level of the one or more volatile biomarkers in the sample to a reference value. A difference in the level of the one or more volatile biomarkers in the urine sample compared to the reference value is indicative of cancer in the subject. The difference compared to the reference value may be an increase, as defined and exemplified below, or a decrease, as defined and exemplified below.

In one embodiment, a decrease in the level of the one or more biomarkers in the urine sample compared to the reference value is indicative of cancer in the subject. Typically, such a decrease is a result of downregulation of the biomarker. As is known to the person skilled in the art, downregulation is the process by which a cell decreases the quantity of a cellular component in response to an external variable.

In one embodiment, an increase in the level of the one or more biomarkers in the urine sample compared to the reference value is indicative of cancer in the subject. Typically, such a decrease is a result of upregulation of the biomarker. As is known to the person skilled in the art, upregulation is the process by which a cell increases the quantity of a cellular component in response to an external variable.

Typically, the increase or decrease in the level of the one or more biomarkers in the urine sample compared to the reference value is measured as a % mean difference. In this specification the term “% mean difference” means the % difference of the total ion count for each mass ion(s) in subjects having cancer, compared with the total ion count in reference subjects (i.e. controls).

Where the measurement comprises an increase of the total ion count for the appropriate mass ion(s) in subjects having cancer, compared with the reference value, the % mean difference is measured as (mean cancer/mean control)×100%. Where the measurement comprises a decrease of the total ion count for the appropriate mass ion(s) in subjects having cancer, compared with the reference value, the % mean difference is measured as (mean control/mean cancer)×100%. The % mean difference therefore always exceeds 100%, except in the situation where the total ion count for the appropriate mass ion(s) in subjects having cancer is exactly the same as the reference value.

In the embodiments wherein an increase in the level of the one or more biomarkers in the urine sample compared to the reference value is indicative of cancer in the subject, the % mean difference in the level of the one or more biomarkers in the urine sample compared to the reference value is not particularly limited. In one embodiment, the % mean difference is at least 100%, such as at least 101%, such as at least 102%, such as at least 103%, such as at least 104%, such as at least 105%, such as at least 106%, such as at least 107%, such as at least 108%, such as at least 109%, such as at least 110%, such as at least 112%, such as at least 114%, such as at least 116%, such as at least 118%, such as at least 120%, such as at least 130%, such as at least 140%, such as at least 150%, such as at least 160%, such as at least 170%, such as at least 180%, such as at least 190%, such as at least 200%, such as at least 250%, such as at least 300%, such as at least 350%, such as at least 400%, such as at least 450%, such as at least 500%, such as at least 550%, such as at least 600%, such as at least 650%, such as at least 700%, such as at least 750%, such as at least 800%, such as at least 850%, such as at least 900%, such as at least 950%, such as at least 1000%, such as at least 1100%, such as at least 1200%, such as at least 1300%, such as at least 1400%, such as at least 1500%, such as at least 1600%, such as at least 1700%, such as at least 1800%, such as at least 1900%, such as at least 2000%, such as at least 2500%, such as at least 3000%, such as at least 3500%, such as at least 4000%, such as at least 4500%, such as at least 5000%, such as at least 5500%, such as at least 6000%, such as at least 6500%, such as at least 7000%, such as at least 7500%, such as at least 8000%, such as at least 8500%, such as at least 9000%, such as at least 9500%, such as at least 10,000%, such as at least 11,000%, such as at least 12,000%, such as at least 13,000%, such as at least 14,000%, such as at least 15,000%, such as at least 16,000%, such as at least 17,000%, such as at least 18,000%, such as at least 19,000%, such as at least 20,000%, such as at least 25,000%, such as at least 30,000%, such as at least 35,000%, such as at least 40,000%, such as at least 45,000%, such as at least 50,000%, such as at least 55,000%, such as at least 60,000%, such as at least 65,000%, such as at least 70,000%, such as at least 75,000%, such as at least 80,000%, such as at least 85,000%, such as at least 90,000%, such as at least 95,000%, such as at least 100,000%.

In the embodiments wherein an increase in the level of the one or more biomarkers in the urine sample compared to the reference value is indicative of cancer in the subject, the % mean difference is typically from 101% to 15,000%, such as 105% to 12,000%, such as 110% to 10,000%, such as 110% to 9000%, such as 120% to 8000%, such as 130% to 7000%, such as 140% to 6000%, such as 150% to 5000%, such as 160% to 4000%, such as 170% to 3000%, such as 180% to 2500%, such as 190% to 2250%, such as 200% to 2000%, such as 250% to 1900%, such as 300% to 1800%, such as 350% to 1700%, such as 400% to 1600%, such as 450% to 1550%, such as 500% to 1500%. In some embodiments, the % mean difference is 101% to 150%. In some embodiments, the % mean difference is 101% to 200%. In some embodiments, the % mean difference is 105% to 300%. In some embodiments, the % mean difference is 120% to 250%. In some embodiments, the % mean difference is 150% to 300%. In some embodiments, the % mean difference is 105% to 120%. In some embodiments, the % mean difference is 120% to 800%. In some embodiments, the % mean difference is 150% to 200%. In some embodiments, the % mean difference is 120% to 500%.

In the embodiments wherein a decrease in the level of the one or more biomarkers in the urine sample compared to the reference value is indicative of cancer in the subject, the % mean difference in the level of the one or more biomarkers in the urine sample compared to the reference value is not particularly limited. In one embodiment, the % mean difference is at least 100%, such as at least 101%, such as at least 102%, such as at least 103%, such as at least 104%, such as at least 105%, such as at least 106%, such as at least 107%, such as at least 108%, such as at least 109%, such as at least 110%, such as at least 112%, such as at least 114%, such as at least 116%, such as at least 118%, such as at least 120%, such as at least 130%, such as at least 140%, such as at least 150%, such as at least 160%, such as at least 170%, such as at least 180%, such as at least 190%, such as at least 200%, such as at least 250%, such as at least 300%, such as at least 350%, such as at least 400%, such as at least 450%, such as at least 500%, such as at least 550%, such as at least 600%, such as at least 650%, such as at least 700%, such as at least 750%, such as at least 800%, such as at least 850%, such as at least 900%, such as at least 950%, such as at least 1000%, such as at least 1100%, such as at least 1200%, such as at least 1300%, such as at least 1400%, such as at least 1500%, such as at least 1600%, such as at least 1700%, such as at least 1800%, such as at least 1900%, such as at least 2000%, such as at least 2500%, such as at least 3000%, such as at least 3500%, such as at least 4000%, such as at least 4500%, such as at least 5000%, such as at least 5500%, such as at least 6000%, such as at least 6500%, such as at least 7000%, such as at least 7500%, such as at least 8000%, such as at least 8500%, such as at least 9000%, such as at least 9500%, such as at least 10,000%, such as at least 11,000%, such as at least 12,000%, such as at least 13,000%, such as at least 14,000%, such as at least 15,000%, such as at least 16,000%, such as at least 17,000%, such as at least 18,000%, such as at least 19,000%, such as at least 20,000%, such as at least 25,000%, such as at least 30,000%, such as at least 35,000%, such as at least 40,000%, such as at least 45,000%, such as at least 50,000%, such as at least 55,000%, such as at least 60,000%, such as at least 65,000%, such as at least 70,000%, such as at least 75,000%, such as at least 80,000%, such as at least 85,000%, such as at least 90,000%, such as at least 95,000%, such as at least 100,000%.

In the embodiments wherein a decrease in the level of the one or more biomarkers in the urine sample compared to the reference value is indicative of cancer in the subject, the % mean difference is typically from 101% to 15,000%, such as 105% to 12,000%, such as 110% to 10,000%, such as 110% to 9000%, such as 120% to 8000%, such as 130% to 7000%, such as 140% to 6000%, such as 150% to 5000%, such as 160% to 4000%, such as 170% to 3000%, such as 180% to 2500%, such as 190% to 2250%, such as 200% to 2000%, such as 250% to 1900%, such as 300% to 1800%, such as 350% to 1700%, such as 400% to 1600%, such as 450% to 1550%, such as 500% to 1500%. In some embodiments, the % mean difference is 101% to 150%. In some embodiments, the % mean difference is 101% to 200%. In some embodiments, the % mean difference is 105% to 300%. In some embodiments, the % mean difference is 120% to 250%. In some embodiments, the % mean difference is 150% to 300%. In some embodiments, the % mean difference is 105% to 120%. In some embodiments, the % mean difference is 120% to 800%. In some embodiments, the % mean difference is 150% to 200%. In some embodiments, the % mean difference is 120% to 500%.

The method may be used to diagnose a wide variety of cancers in the subject. Examples of cancers include carcinoma such as cancer of the bladder, breast, colon, kidney, liver, lung (including non-small cell cancer and small cell lung cancer), oesophagus, gall-bladder, ovary, pancreas, stomach, cervix, thyroid, prostate, skin, squamous cell carcinoma, testis, genitourinary tract, larynx, glioblastoma, neuroblastoma, keratoacanthoma, epidermoid carcinoma, large cell carcinoma, non-small cell lung carcinoma, small cell lung carcinoma, lung adenocarcinoma, bone, adenoma, adenocarcinoma, follicular carcinoma, undifferentiated carcinoma, papilliary carcinoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukaemia; hematopoietic tumours of lymphoid lineage, including leukaemia, acute lymphocitic leukaemia, acute lymphoblastic leukaemia, B-cell lymphoma, T-cell-lymphoma, Hodgkin's lymphoma, non-Hodgkin's lymphoma, hairy cell lymphoma and Burkett's lymphoma; hematopoietic tumours of myeloid lineage, including acute and chronic myelogenous leukaemias, myelodysplastic syndrome and promyelocytic leukaemia; tumours of mesenchymal origin, including fibrosarcoma and rhabdomyosarcoma; tumours of the central and peripheral nervous system, including astrocytoma, neuroblastoma, glioma and schwannomas; and other tumours, including melanoma, seminoma, teratocarcinoma, osteosarcoma, xeroderma pigmentosum, keratoxanthoma, thyroid follicular cancer and Kaposi's sarcoma.

Preferably, the cancer is selected from bladder, renal, lung, prostate, pancreatic, colorectal or breast cancer.

Cancers and Specific Biomarkers Identified

The present invention also relates to a method of diagnosing cancer in a subject comprising determining a level of a biomarker for cancer in a urine sample obtained from the subject. In contrast to the methods described in the prior art, which did not identify the specific biomarkers, the mass spectrometry method of the present invention allows both the identification of the specific compounds which act as biomarkers and their quantification (in terms of % mean difference from control subjects), thereby enabling both more rapid and more precise diagnosis and treatment of the cancer.

Without wishing to be bound by theory, it is believed that the % mean difference of a biomarker (or combination of biomarkers) in cancer patients compared with controls may act as a “fingerprint” for diagnosis, having the potential to identify each cancer with its own particular biomarker or combination thereof.

The method of the present invention also confers the potential for a doctor to identify whether a treatment has been successful, in that a successful treatment could be identified by a regression to the mean of the specific biomarker compared with the measured value prior to treatment.

Typically, the biomarker is a volatile organic compound (as defined and exemplified above) having a mass/charge ratio (m/z) as defined herein. In one embodiment, the biomarker has a m/z of 16 to 290. In one embodiment, the biomarker has a m/z of 32 to 290. In one embodiment, the biomarker has a m/z of 32 to 250. In one embodiment, the biomarker has a m/z of 32 to 210. In one embodiment, the biomarker has a m/z of 32 to 206. In one embodiment, the biomarker has a m/z of 50 to 200. In one embodiment, the biomarker has a m/z of 70 to 180. In one embodiment, the biomarker has a m/z of 90 to 160.

In one embodiment, the biomarker has a m/z of 32 to 40. In one embodiment, the biomarker has a m/z of 40 to 50. In one embodiment, the biomarker has a m/z of 50 to 60. In one embodiment, the biomarker has a m/z of 60 to 70. In one embodiment, the biomarker has a m/z of 70 to 80. In one embodiment, the biomarker has a m/z of 80 to 90. In one embodiment, the biomarker has a m/z of 90 to 100. In one embodiment, the biomarker has a m/z of 100 to 110. In one embodiment, the biomarker has a m/z of 110 to 120. In one embodiment, the biomarker has a m/z of 120 to 130. In one embodiment, the biomarker has a m/z of 130 to 140. In one embodiment, the biomarker has a m/z of 140 to 150. In one embodiment, the biomarker has a m/z of 150 to 160. In one embodiment, the biomarker has a m/z of 160 to 170. In one embodiment, the biomarker has a m/z of 170 to 180. In one embodiment, the biomarker has a m/z of 180 to 190. In one embodiment, the biomarker has a m/z of 190 to 200. In one embodiment, the biomarker has a m/z of 200 to 210.

In one embodiment, the biomarker has a m/z of 134 and a H⁺ pseudo molecular ion of m/z of 135. In one embodiment, the biomarker has a m/z of 186 and a H⁺ pseudo molecular ion of m/z of 187. In one embodiment, the biomarker has a m/z of 206 and a H⁺ pseudo molecular ion of m/z of 207. In one embodiment, the biomarker has a m/z of 156 and a H⁺ pseudo molecular ion of m/z of 157. In one embodiment, the biomarker has a m/z of 81 and a H⁺ pseudo molecular ion of m/z of 82. In one embodiment, the biomarker has a m/z of 128 and a H⁺ pseudo molecular ion of m/z of 129. In one embodiment, the biomarker has a m/z of 126 and a H⁺ pseudo molecular ion of m/z of 127. In one embodiment, the biomarker has a m/z of 114 and a H⁺ pseudo molecular ion of m/z of 115. In one embodiment, the biomarker has a m/z of 142 and a H⁺ pseudo molecular ion of m/z of 143. In one embodiment, the biomarker has a m/z of 156 and a H⁺ pseudo molecular ion of m/z of 157. In one embodiment, the biomarker has a m/z of 128 and a H⁺ pseudo molecular ion of m/z of 129. In one embodiment, the biomarker has a m/z of 114 and a H⁺ pseudo molecular ion of m/z of 115. In one embodiment, the biomarker has a m/z of 147 and a H⁺ pseudo molecular ion of m/z of 148. In one embodiment, the biomarker has a m/z of 150 and a H⁺ pseudo molecular ion of m/z of 151. In one embodiment, the biomarker has a m/z of 86 and a H⁺ pseudo molecular ion of m/z of 87. In one embodiment, the biomarker has a m/z of 108 and a H⁺ pseudo molecular ion of m/z of 109. In one embodiment, the biomarker has a m/z of 45 and a H⁺ pseudo molecular ion of m/z of 46. In one embodiment, the biomarker has a m/z of 112 and a H⁺ pseudo molecular ion of m/z of 113. In one embodiment, the biomarker has a m/z of 152 and a H⁺ pseudo molecular ion of m/z of 153. In one embodiment, the biomarker has a m/z of 196 and a H⁺ pseudo molecular ion of m/z of 197. In one embodiment, the biomarker has a m/z of 46 and a H⁺ pseudo molecular ion of m/z of 47. In one embodiment, the biomarker has a m/z of 58 and a H⁺ pseudo molecular ion of m/z of 59. In one embodiment, the biomarker has a m/z of 110 and a H⁺ pseudo molecular ion of m/z of 111. In one embodiment, the biomarker has a m/z of 32 and a H⁺ pseudo molecular ion of m/z of 33. In one embodiment, the biomarker has a m/z of 152 and a H⁺ pseudo molecular ion of m/z of 153. In one embodiment, the biomarker has a m/z of 98 and a H⁺ pseudo molecular ion of m/z of 99. In one embodiment, the biomarker has a m/z of 94 and a H⁺ pseudo molecular ion of m/z of 95. In one embodiment, the biomarker has a m/z of 120 and a H⁺ pseudo molecular ion of m/z of 121. In one embodiment, the biomarker has a m/z of 116 and a H⁺ pseudo molecular ion of m/z of 117. Herein the term “H⁺ pseudo molecular ion” is synonymous with the ion [M+H]⁺.

In one embodiment, the biomarker does not have a m/z of 29 to 31. In one embodiment, the biomarker does not have a m/z of 30.

In one embodiment, the biomarker is an alcohol, typically a C₁₋₂₀ alcohol, preferably a C₁₋₁₄ alcohol. Particular alcohol biomarkers identified herein include methanol, ethanol, 2-ethyl-1-hexanol, 1-octanol, 1-dodecanol and menthol.

In one embodiment, the biomarker is an aldehyde, typically a C₂₋₂₀ aldehyde, preferably a C₂₋₁₄ aldehyde. Particular aldehyde biomarkers identified herein include heptanal, octanal, nonanal, ethyl benzaldehydes (a defined and exemplified below) and dimethyl benzaldehydes (a defined and exemplified below).

In one embodiment, the biomarker is an ester, typically a C₂₋₂₀ ester, preferably a C₂₋₁₄ ester (the carbon number being the total number of carbons on the alkyl and acyl part). Particular ester biomarkers identified herein include butyl acetate.

In one embodiment, the biomarker is a ketone, typically a C₃₋₂₀ ketone, preferably a C₃₋₁₄ ketone, and more preferably a C₆₋₁₂ ketone. Particular ketone biomarkers identified herein include acetone, 2-butanone, 2-pentanone, 2-heptanone, 4-heptanone, 2-octanone, 3-octen-2-one, 2-nonanone, 2-decanone, cyclohexanone, acetophenone and piperitone.

In one embodiment, the biomarker is a phenol, the phenol being optionally substituted on the aromatic ring by 1 to 3 C₁₋₄ alkyl groups. Particular phenol biomarkers identified herein include phenol and 2,4-bis(1,1-dimethylethyl)phenol.

In one embodiment, the biomarker is a nitrile, typically a C₃₋₁₀ nitrile. Particular nitrile biomarkers identified herein include 2-methyl-2-butenenitrile.

In one embodiment, the biomarker is an alkene, typically a C₂₋₂₀ alkene, preferably a C₄₋₁₆ aldehyde. Particular alkene biomarkers identified herein include 1-tetradecene.

In one embodiment, the biomarker is an aromatic hydrocarbon, the aromatic hydrocarbon being optionally substituted on the aromatic ring by 1 to 3 C₁₋₄ alkyl groups. Particular aromatic hydrocarbon biomarkers identified herein include benzene.

In one embodiment, the biomarker is a heteroaromatic compound having 1 to 5 nitrogen atoms and, optionally 1 oxygen atom and 1 sulphur atom, in 1 or 2 rings, the heteroaromatic hydrocarbon being optionally substituted on the heteroaromatic ring by 1 to 3 C₁₋₄ alkyl groups. Particular heteroaromatic biomarkers identified herein include 2,5-dimethylpyrazine and 3-ethyl[1,2,4]triazolo[4,3-a]pyridine.

In one embodiment, the biomarker is a mono, di- or trialkylamine, typically a mono-(C₁₋₆ alkyl)amine, a di-(C₁₋₆ alkyl)amine or a tri-(C₁₋₆ alkyl)amine. In di-(C₁₋₆ alkyl)amine or a tri-(C₁₋₆ alkyl)amine, the alkyl groups may be the same or different. Particular amine biomarkers identified herein include dimethylamine (N-methylmethanamine).

According to the present invention, the level of the specific biomarkers and combinations thereof disclosed herein is determined by a method comprising mass spectrometry. The mass spectrometry method may be any of those described and exemplified above.

In one embodiment, the mass spectrometry is tandem mass spectrometry. According to the present invention, carrying out the analysis using tandem mass spectrometry enables both the parent and daughter ions to be analysed, therefore improving the accuracy of the technique and allowing the differentiation of molecular ions having the same m/z ratio.

The mass spectrometry method may be any of those described and exemplified above. In one embodiment, the tandem mass spectrometry is ion trap mass spectrometry. In one embodiment, the tandem mass spectrometry is quadrupole time-of-flight mass spectrometry. In one embodiment, the tandem mass spectrometry is triple quadrupole mass spectrometry. In one embodiment, the tandem mass spectrometry is Quadrupole-Ion Traps mass spectrometry. In one embodiment, the tandem mass spectrometry is Ion Mobility Spectrometer-Quadrupole Ion Traps mass spectrometry. In one embodiment, the tandem mass spectrometry is Time of Flight, Ion Trap-Fourier Transform mass spectrometry. In one embodiment, the tandem mass spectrometry is Secondary Electrospray Ionisation mass spectrometry.

In one embodiment, the mass spectrometry is used in tandem with a chromatographic separation technique. In one embodiment, the method is GC-MS. In one embodiment, the method is LC-MS.

In one embodiment, the mass spectrometry is carried out without a prior chromatographic step. In one embodiment, the mass spectrometry is direct infusion mass spectrometry. In one embodiment, the mass spectrometry is direct sampling mass spectrometry.

Without wishing to be bound by theory, it is believed that one or more of the biomarkers identified using the claimed methods may be involved in the metabolic pathway of the cancer. The method therefore confers the potential for supplementation or deprivation of the specified biomarker in order to reverse this step in the pathway and treat the cancer.

In particular, it has been surprisingly found by the present inventors that in a wide range of cancer patients (particularly although not exclusively patients having cancer selected from bladder, renal, lung, prostate, pancreatic or breast cancer), levels of an ethylbenzaldehyde or a dimethylbenzaldehyde are lower than in control patients. Therefore, a decrease in the level of an ethylbenzaldehyde or a dimethylbenzaldehyde in the urine sample compared to a reference value is indicative of cancer (particularly although not exclusively bladder, renal, lung, prostate, pancreatic or breast cancer) in the subject.

In one embodiment the ethylbenzaldehyde is 2-ethylbenzaldehyde (o-ethylbenzaldehyde). In one embodiment the ethylbenzaldehyde is 3-ethylbenzaldehyde (m-ethylbenzaldehyde). In one embodiment the ethylbenzaldehyde is 4-ethylbenzaldehyde (p-ethylbenzaldehyde).

In one embodiment the dimethylbenzaldehyde is 2,3-dimethylbenzaldehyde. In one embodiment the dimethylbenzaldehyde is 2,4-dimethylbenzaldehyde. In one embodiment the dimethylbenzaldehyde is 2,5-dimethylbenzaldehyde. In one embodiment the dimethylbenzaldehyde is 2,6-dimethylbenzaldehyde. In one embodiment the dimethylbenzaldehyde is 3,4-dimethylbenzaldehyde. In one embodiment the dimethylbenzaldehyde is 3,5-dimethylbenzaldehyde.

Therefore, in a further aspect the invention provides a method of diagnosing cancer in a subject comprising determining a level of a biomarker for cancer in a urine sample obtained from the subject, wherein the biomarker is an ethylbenzaldehyde or a dimethylbenzaldehyde. Preferably, the method further comprises comparing the level of an ethylbenzaldehyde or a dimethylbenzaldehyde in the urine sample to a reference value (i.e. a control value), wherein the level of the ethylbenzaldehyde or the dimethylbenzaldehyde in the urine sample compared to the reference value is indicative of cancer in the subject. More preferably, a decrease in the level of an ethylbenzaldehyde or a dimethylbenzaldehyde in the urine sample compared to the reference value is indicative of cancer in the subject. The % mean difference in the level of the ethylbenzaldehyde or the dimethylbenzaldehyde in the urine sample compared to the reference value is at least 100%, such as at least 200%, such as at least 300%, such as at least 400%, such as at least 500%, such as at least 600%, such as at least 700%, such as at least 800%, such as at least 900%, such as at least 1000%, such as at least 1500%, such as at least 2000%, such as at least 3000%, such as at least 4000%, such as at least 5000%, such as at least 6000%, such as at least 7000%, such as at least 8000%, such as at least 9000%, such as at least 10,000%, such as at least 15,000%, such as at least 20,000%, such as at least 30,000%, such as at least 40,000%, such as at least 50,000%, such as at least 60,000%, such as at least 70,000%, such as at least 80,000%, such as at least 90,000%, such as at least 100,000%.

In one embodiment, a decrease in the level of the ethylbenzaldehyde or the dimethylbenzaldehyde in the urine sample compared to the reference value is indicative of cancer in the subject and the % mean difference in the level of the ethylbenzaldehyde or the dimethylbenzaldehyde in the urine sample compared to the reference value is from 100% to 20,000%. In one embodiment, a decrease in the level of the ethylbenzaldehyde or the dimethylbenzaldehyde in the urine sample compared to the reference value is indicative of cancer in the subject and the % mean difference in the level of the ethylbenzaldehyde or the dimethylbenzaldehyde in the urine sample compared to the reference value is from 200% to 15,000%. In one embodiment, a decrease in the level of the ethylbenzaldehyde or the dimethylbenzaldehyde in the urine sample compared to the reference value is indicative of cancer in the subject and the % mean difference in the level of the ethylbenzaldehyde or the dimethylbenzaldehyde in the urine sample compared to the reference value is from 10,000% to 15,000%. In one embodiment, a decrease in the level of the ethylbenzaldehyde or the dimethylbenzaldehyde in the urine sample compared to the reference value is indicative of cancer in the subject and the % mean difference in the level of the ethylbenzaldehyde or the dimethylbenzaldehyde in the urine sample compared to the reference value is from 1000% to 1500%. In one embodiment, a decrease in the level of the ethylbenzaldehyde or the dimethylbenzaldehyde in the urine sample compared to the reference value is indicative of cancer in the subject and the % mean difference in the level of the ethylbenzaldehyde or the dimethylbenzaldehyde in the urine sample compared to the reference value is from 1500% to 2000%. In one embodiment, a decrease in the level of the ethylbenzaldehyde or the dimethylbenzaldehyde in the urine sample compared to the reference value is indicative of cancer in the subject and the % mean difference in the level of the ethylbenzaldehyde or the dimethylbenzaldehyde in the urine sample compared to the reference value is from 300% to 400%.

In one embodiment, the cancer is pancreatic cancer, a decrease in the level of an ethylbenzaldehyde or a dimethylbenzaldehyde in the urine sample compared to the reference value is indicative of pancreatic cancer in the subject and the and the % mean difference in the level of the ethylbenzaldehyde or the dimethylbenzaldehyde in the urine sample of the pancreatic cancer patient compared to the reference value is typically from 5000% to 50,000%, preferably from 10,000% to 15,000%.

In one embodiment, the cancer is lung cancer, a decrease in the level of an ethylbenzaldehyde or a dimethylbenzaldehyde in the urine sample compared to the reference value is indicative of lung cancer in the subject and the % mean difference in the level of the ethylbenzaldehyde or the dimethylbenzaldehyde in the urine sample of the lung cancer patient compared to the reference value is typically from 500% to 5000%, preferably from 1000% to 1500%.

In one embodiment, the cancer is breast cancer, a decrease in the level of the ethylbenzaldehyde or the dimethylbenzaldehyde in the urine sample compared to the reference value is indicative of breast cancer in the subject and the % mean difference in the level of the ethylbenzaldehyde or the dimethylbenzaldehyde in the urine sample of the breast cancer patient compared to the reference value compared to the reference value is typically from 400% to 4000%, preferably from 1500% to 2000%.

In one embodiment, the cancer is prostate cancer, a decrease in the level of an ethylbenzaldehyde or a dimethylbenzaldehyde in the urine sample compared to the reference value is indicative of prostate cancer in the subject and the % mean difference in the level of the ethylbenzaldehyde or the dimethylbenzaldehyde in the urine sample of the prostate cancer patient compared to the reference value is typically from 400% to 4000%, preferably from 1500% to 2000%.

In one embodiment, the cancer is renal cancer, a decrease in the level of an ethylbenzaldehyde or a dimethylbenzaldehyde in the urine sample compared to the reference value is indicative of renal cancer in the subject and the % mean difference in the level of the ethylbenzaldehyde or the dimethylbenzaldehyde in the urine sample of the renal cancer patient compared to the reference value is typically from 200% to 800%, preferably from 300% to 400%.

In one embodiment, the cancer is bladder cancer, a decrease in the level of an ethylbenzaldehyde or a dimethylbenzaldehyde in the urine sample compared to the reference value is indicative of bladder cancer in the subject and the % mean difference in the level of the ethylbenzaldehyde or the dimethylbenzaldehyde in the urine sample of the bladder cancer patient compared to the reference value is typically from 200% to 800%, preferably from 300% to 400%.

It has also been found by the present inventors that in patients having specific cancers, particularly from bladder, renal, lung, prostate, pancreatic, colorectal or breast cancer, measured levels of other specific biomarkers differ from those measured in reference (control) patients. The difference may be an increase or a decrease, depending on the particular biomarker being measured and the particular cancer. Therefore, an increase or a decrease in the level of these specific biomarkers in the urine sample of the patient compared to a reference (control) value may be indicative of this specific cancer in the subject.

In one embodiment, the cancer is pancreatic cancer, the biomarker has an m/z of 186 and a H⁺ pseudo molecular ion of m/z of 187 and a decrease in the level of this biomarker in the urine sample compared to the reference value is indicative of pancreatic cancer in the subject. In this embodiment the biomarker is preferably 1-dodecanol. In this embodiment, the % mean difference in the level of this biomarker in the urine sample of the pancreatic cancer patient compared to the reference value is typically from 1000% to 10,000%, preferably from 3000% to 4000%. In one embodiment, the cancer is pancreatic cancer, the biomarker has an m/z of 206 and a H⁺ pseudo molecular ion of m/z of 207 and a decrease in the level of this biomarker in the urine sample compared to the reference value is indicative of pancreatic cancer in the subject. In this embodiment, the biomarker is preferably 2,4-bis(1,1-dimethylethyl)phenol. In this embodiment, the % mean difference in the level of this biomarker in the urine sample of the pancreatic cancer patient compared to the reference value is typically from 100% to 1000%, preferably from 200% to 400%.

In one embodiment, the cancer is pancreatic cancer, the biomarker has an m/z of 156 and a H⁺ pseudo molecular ion of m/z of 157 and a decrease in the level of this biomarker in the urine sample compared to the reference value is indicative of pancreatic cancer in the subject. In this embodiment, the biomarker is preferably menthol. In this embodiment, the % mean difference in the level of this biomarker in the urine sample of the pancreatic cancer patient compared to the reference value is typically from 200% to 2000%, preferably from 600% to 800%.

In one embodiment, the cancer is pancreatic cancer, the biomarker has an m/z of 81 and a H⁺ pseudo molecular ion of m/z of 82 and a decrease in the level of this biomarker in the urine sample compared to the reference value is indicative of pancreatic cancer in the subject. In this embodiment, the biomarker is preferably 2-methyl-2-butenenitrile. In this embodiment, the % mean difference in the level of this biomarker in the urine sample of the pancreatic cancer patient compared to the reference value is typically from 5,000% to 50,000%, preferably from 10,000% to 15,000%.

In one embodiment, the cancer is pancreatic cancer, the biomarker has an m/z of 128 and a H⁺ pseudo molecular ion of m/z of 129 and an increase in the level of this biomarker in the urine sample compared to the reference value is indicative of pancreatic cancer in the subject. In this embodiment, the biomarker is preferably octanal. In this embodiment, the % mean difference in the level of this biomarker in the urine sample of the pancreatic cancer patient compared to the reference value is typically from 500% to 5000%, preferably from 1000% to 2000%.

In one embodiment, the cancer is pancreatic cancer, the biomarker has an m/z of 114 and a H⁺ pseudo molecular ion of m/z of 115 and an increase in the level of this biomarker in the urine sample compared to the reference value is indicative of pancreatic cancer in the subject. In this embodiment, the biomarker is preferably heptanal. In this embodiment, the % mean difference in the level of this biomarker in the urine sample of the pancreatic cancer patient compared to the reference value is typically from 400% to 4000%, preferably from 600% to 1000%.

In one embodiment, the cancer is pancreatic cancer, the biomarker has an m/z of 142 and a H⁺ pseudo molecular ion of m/z of 143 and an increase in the level of this biomarker in the urine sample compared to the reference value is indicative of pancreatic cancer in the subject. In this embodiment, the biomarker is preferably nonanal. In this embodiment, the % mean difference in the level of this biomarker in the urine sample of the pancreatic cancer patient compared to the reference value is typically from 200% to 800%, preferably from 300% to 500%.

In one embodiment, the cancer is pancreatic cancer, the biomarker has an m/z of 142 and a H⁺ pseudo molecular ion of m/z of 143 and an increase in the level of this biomarker in the urine sample compared to the reference value is indicative of pancreatic cancer in the subject. In this embodiment, the biomarker is preferably 2-nonanone. In this embodiment, the % mean difference in the level of this biomarker in the urine sample of the pancreatic cancer patient compared to the reference value is typically from 1000% to 10,000%, preferably from 2000% to 4000%.

In one embodiment, the cancer is pancreatic cancer, the biomarker has an m/z of 156 and a H⁺ pseudo molecular ion of m/z of 157 and an increase in the level of this biomarker in the urine sample compared to the reference value is indicative of pancreatic cancer in the subject. In this embodiment, the biomarker is preferably 2-decanone. In this embodiment, the % mean difference in the level of this biomarker in the urine sample of the pancreatic cancer patient compared to the reference value is typically from 2000% to 20,000%, preferably from 3000% to 7000%.

In one embodiment, the cancer is pancreatic cancer, the biomarker has an m/z of 128 and a H⁺ pseudo molecular ion of m/z of 129 and an increase in the level of this biomarker in the urine sample compared to the reference value is indicative of pancreatic cancer in the subject. In this embodiment, the biomarker is preferably 2-octanone. In this embodiment, the % mean difference in the level of this biomarker in the urine sample of the pancreatic cancer patient compared to the reference value is typically from 500% to 5000%, preferably from 600% to 15,000%.

In one embodiment, the cancer is pancreatic cancer, the biomarker has an m/z of 142 and a H⁺ pseudo molecular ion of m/z of 143 and an increase in the level of this biomarker in the urine sample compared to the reference value is indicative of pancreatic cancer in the subject. In this embodiment, the biomarker is preferably nonanal. In this embodiment, the % mean difference in the level of this biomarker in the urine sample of the pancreatic cancer patient compared to the reference value is typically from 101% to 300%, preferably from 105% to 200%.

In one embodiment, the cancer is pancreatic cancer, the biomarker has an m/z of 114 and a H⁺ pseudo molecular ion of m/z of 115 and a decrease in the level of this biomarker in the urine sample compared to the reference value is indicative of pancreatic cancer in the subject. In this embodiment, the biomarker is preferably 2-heptanone. In this embodiment, the % mean difference in the level of this biomarker in the urine sample of the pancreatic cancer patient compared to the reference value is typically from 105% to 500%, preferably from 110% to 250%.

In one embodiment, the cancer is pancreatic cancer, the biomarker has an m/z of 114 and a H⁺ pseudo molecular ion of m/z of 115 and a decrease in the level of this biomarker in the urine sample compared to the reference value is indicative of pancreatic cancer in the subject. In this embodiment, the biomarker is preferably 4-heptanone. In this embodiment, the % mean difference in the level of this biomarker in the urine sample of the pancreatic cancer patient compared to the reference value is typically from 101% to 300%, preferably from 105% to 200%. In one embodiment, the cancer is lung cancer, the biomarker has an m/z of 206 and a H⁺ pseudo molecular ion of m/z of 207 and a decrease in the level of this biomarker in the urine sample compared to the reference value is indicative of lung cancer in the subject. In this embodiment, the biomarker is preferably 2,4-bis(1,1-dimethylethyl)phenol. In this embodiment, the % mean difference in the level of this biomarker in the urine sample of the lung cancer patient compared to the reference value is typically from 150% to 900%, preferably from 300% to 500%.

In one embodiment, the cancer is lung cancer, the biomarker has an m/z of 186 and a H⁺ pseudo molecular ion of m/z of 187 is 1-dodecanol and a decrease in the level of this biomarker in the urine sample compared to the reference value is indicative of lung cancer in the subject. In this embodiment, the biomarker is preferably 1-dodecanol. In this embodiment, the % mean difference in the level of this biomarker in the urine sample of the lung cancer patient compared to the reference value is typically from 500% to 5000%, preferably from 1500% to 2500%.

In one embodiment, the cancer is lung cancer, the biomarker has an m/z of 147 and a H⁺ pseudo molecular ion of m/z of 148 and a decrease in the level of this biomarker in the urine sample compared to the reference value is indicative of lung cancer in the subject. In this embodiment, the biomarker is preferably -ethyl[1,2,4]triazolo[4,3-a]pyridine In this embodiment, the % mean difference in the level of this biomarker in the urine sample of the lung cancer patient compared to the reference value is typically from 600% to 6000%, preferably from 2000% to 4000%.

In one embodiment, the cancer is lung cancer, the biomarker has an m/z of 150 and a H⁺ pseudo molecular ion of m/z of 151 and a decrease in the level of this biomarker in the urine sample compared to the reference value is indicative of lung cancer in the subject. In this embodiment, the biomarker is preferably an oxygenated sesquiterpene. In this embodiment, the % mean difference in the level of this biomarker in the urine sample of the lung cancer patient compared to the reference value is typically from 100% to 150%, preferably from 100% to 110%.

In one embodiment, the cancer is lung cancer, the biomarker has an m/z of 86 and a H⁺ pseudo molecular ion of m/z of 87 and a decrease in the level of this biomarker in the urine sample compared to the reference value is indicative of lung cancer in the subject. In this embodiment, the biomarker is preferably 2-pentanone. In this embodiment, the % mean difference in the level of this biomarker in the urine sample of the lung cancer patient compared to the reference value is typically from 120% to 250%, preferably from 150% to 200%.

In one embodiment, the cancer is lung cancer, the biomarker has an m/z of 142 and a H⁺ pseudo molecular ion of m/z of 143 and an increase in the level of this biomarker in the urine sample compared to the reference value is indicative of lung cancer in the subject. In this embodiment, the biomarker is preferably nonanal. In this embodiment, the % mean difference in the level of this biomarker in the urine sample of the lung cancer patient compared to the reference value is typically from 500% to 5000%, preferably from 1500% to 2500%.

In one embodiment, the cancer is lung cancer, the biomarker has an m/z of 142 and a H⁺ pseudo molecular ion of m/z of 143 and an increase in the level of this biomarker in the urine sample compared to the reference value is indicative of lung cancer in the subject. In this embodiment, the biomarker is preferably 2-nonanone. In this embodiment, the % mean difference in the level of this biomarker in the urine sample of the lung cancer patient compared to the reference value is typically from 600% to 6000%, preferably from 1500% to 3000%.

In one embodiment, the cancer is lung cancer, the biomarker has an m/z of 128 and a H⁺ pseudo molecular ion of m/z of 129 and an increase in the level of this biomarker in the urine sample compared to the reference value is indicative of lung cancer in the subject. In this embodiment, the biomarker is preferably octanal. In this embodiment, the % mean difference in the level of this biomarker in the urine sample of the lung cancer patient compared to the reference value is typically from 500% to 5000%, preferably from 1500% to 2500%.

In one embodiment, the cancer is lung cancer, the biomarker has an m/z of 108 and a H⁺ pseudo molecular ion of m/z of 109 and an increase in the level of this biomarker in the urine sample compared to the reference value is indicative of lung cancer in the subject. In this embodiment, the biomarker is preferably 2,5-dimethylpyrazine. In this embodiment, the % mean difference in the level of this biomarker in the urine sample of the lung cancer patient compared to the reference value is typically from 120% to 600%, preferably from 150% to 300%.

In one embodiment, the cancer is lung cancer, the biomarker has an m/z of 45 and a H⁺ pseudo molecular ion of m/z of 46 and an increase in the level of this biomarker in the urine sample compared to the reference value is indicative of lung cancer in the subject. In this embodiment, the biomarker is preferably N-methyl-methanamine (dimethylamine). In this embodiment, the % mean difference in the level of this biomarker in the urine sample of the lung cancer patient compared to the reference value is typically from 110% to 250%, preferably from 120% to 200%.

In one embodiment, the cancer is lung cancer, the biomarker has an m/z of 78 and a H⁺ pseudo molecular ion of m/z of 79 and an increase in the level of this biomarker in the urine sample compared to the reference value is indicative of lung cancer in the subject. In this embodiment, the biomarker is preferably benzene. In this embodiment, the % mean difference in the level of this biomarker in the urine sample of the lung cancer patient compared to the reference value is typically from 150% to 2500%, preferably from 200% to 1000%.

In one embodiment, the cancer is lung cancer, the biomarker has an m/z of 130 and a H⁺ pseudo molecular ion of m/z of 131 and a decrease in the level of this biomarker in the urine sample compared to the reference value is indicative of lung cancer in the subject. In this embodiment, the biomarker is preferably 2-ethyl-1-hexanol. In this embodiment, the % mean difference in the level of this biomarker in the urine sample of the lung cancer patient compared to the reference value is typically from 110% to 250%, preferably from 120% to 200%.

In one embodiment, the cancer is lung cancer, the biomarker has an m/z of 114 and a H⁺ pseudo molecular ion of m/z of 115 and a decrease in the level of this biomarker in the urine sample compared to the reference value is indicative of lung cancer in the subject. In this embodiment, the biomarker is preferably 2-heptanone. In this embodiment, the % mean difference in the level of this biomarker in the urine sample of the lung cancer patient compared to the reference value is typically from 101% to 150%, preferably from 103% to 120%.

In one embodiment, the cancer is breast cancer, the biomarker has an m/z of 112 and a H⁺ pseudo molecular ion of m/z of 113 and an increase in the level of this biomarker in the urine sample compared to the reference value is indicative of breast cancer in the subject. In this embodiment, the biomarker is preferably 3-ethyl-cyclopentanone. In this embodiment, the % mean difference in the level of this biomarker in the urine sample of the breast cancer patient compared to the reference value is typically from 150% to 400%, preferably from 180% to 250%.

In one embodiment, the cancer is breast cancer, the biomarker has an m/z of 152 and a H⁺ pseudo molecular ion of m/z of 153 and an increase in the level of this biomarker in the urine sample compared to the reference value is indicative of breast cancer in the subject. In this embodiment, the biomarker is preferably piperitone. In this embodiment, the % mean difference in the level of this biomarker in the urine sample of the breast cancer patient compared to the reference value is typically from 200% to 500%, preferably from 250% to 400%.

In one embodiment, the cancer is breast cancer, the biomarker has an m/z of 186 and a H⁺ pseudo molecular ion of m/z of 187 and a decrease in the level of this biomarker in the urine sample compared to the reference value is indicative of breast cancer in the subject. In this embodiment, the biomarker is preferably is 1-dodecanol. In this embodiment, the % mean difference in the level of this biomarker in the urine sample of the breast cancer patient compared to the reference value is typically from 500% to 5000%, preferably from 1500% to 2500%.

In one embodiment, the cancer is breast cancer, the biomarker has an m/z of 147 and a H⁺ pseudo molecular ion of m/z of 148 and a decrease in the level of this biomarker in the urine sample compared to the reference value is indicative of breast cancer in the subject. In this embodiment, the biomarker is preferably 3-ethyl[1,2,4]triazolo[4,3-a]pyridine. In this embodiment, the % mean difference in the level of this biomarker in the urine sample of the breast cancer patient compared to the reference value is typically from 800% to 8000%, preferably from 2500% to 5000%.

In one embodiment, the cancer is breast cancer, the biomarker has an m/z of 196 and a H⁺ pseudo molecular ion of m/z of 197 and a decrease in the level of this biomarker in the urine sample compared to the reference value is indicative of breast cancer in the subject. In this embodiment, the biomarker is preferably 1-tetradecene. In this embodiment, the % mean difference in the level of this biomarker in the urine sample of the breast cancer patient compared to the reference value is typically from 500% to 5000%, preferably from 1000% to 3000%.

In one embodiment, the cancer is prostate cancer, the biomarker has an m/z of 150 and a H⁺ pseudo molecular ion of m/z of 151 and an increase in the level of this biomarker in the urine sample compared to the reference value is indicative of prostate cancer in the subject. In this embodiment, the biomarker is preferably an oxygenated sesquiterpene. In this embodiment, the % mean difference in the level of this biomarker in the urine sample of the prostate cancer patient compared to the reference value is typically from 500% to 5000%, preferably from 1000% to 3000%.

In one embodiment, the cancer is prostate cancer, the biomarker has an m/z of 156 and a H⁺ pseudo molecular ion of m/z of 157 and an increase in the level of this biomarker in the urine sample compared to the reference value is indicative of prostate cancer in the subject. In this embodiment, the biomarker is preferably menthol. In this embodiment, the % mean difference in the level of this biomarker in the urine sample of the prostate cancer patient compared to the reference value is typically from 200% to 2000%, preferably from 300% to 900%.

In one embodiment, the cancer is prostate cancer, the biomarker has an m/z of 206 and a H⁺ pseudo molecular ion of m/z of 207 and an increase in the level of this biomarker in the urine sample compared to the reference value is indicative of prostate cancer in the subject. In this embodiment, the biomarker is preferably 2,4-bis(1,1-dimethylethyl)phenol In this embodiment, the % mean difference in the level of this biomarker in the urine sample of the prostate cancer patient compared to the reference value is typically from 100% to 300%, preferably from 120% to 200%.

In one embodiment, the cancer is prostate cancer, the biomarker has an m/z of 46 and a H⁺ pseudo molecular ion of m/z of 47 and a decrease in the level of this biomarker in the urine sample compared to the reference value is indicative of prostate cancer in the subject. In this embodiment, the biomarker is preferably ethanol. In this embodiment, the % mean difference in the level of this biomarker in the urine sample of the prostate cancer patient compared to the reference value is typically from 120% to 500%, preferably from 150% to 250%.

In one embodiment, the cancer is prostate cancer, the biomarker has an m/z of 58 and a H⁺ pseudo molecular ion of m/z of 59 and a decrease in the level of this biomarker in the urine sample compared to the reference value is indicative of prostate cancer in the subject. In this embodiment, the biomarker is preferably acetone. In this embodiment, the % mean difference in the level of this biomarker in the urine sample of the prostate cancer patient compared to the reference value is typically from 120% to 500%, preferably from 150% to 250%.

In one embodiment, the cancer is prostate cancer, the biomarker has an m/z of 186 and a H⁺ pseudo molecular ion of m/z of 187 and a decrease in the level of this biomarker in the urine sample compared to the reference value is indicative of prostate cancer in the subject. In this embodiment, the biomarker is preferably 1-dodecanol. In this embodiment, the % mean difference in the level of this biomarker in the urine sample of the prostate cancer patient compared to the reference value is typically from 500% to 5000%, preferably from 1500% to 3000%.

In one embodiment, the cancer is prostate cancer, the biomarker has an m/z of 142 and a H⁺ pseudo molecular ion of m/z of 143 and an increase in the level of this biomarker in the urine sample compared to the reference value is indicative of prostate cancer in the subject. In this embodiment, the biomarker is preferably 2,4-bis(1,1-dimethylethyl)phenol In this embodiment, the % mean difference in the level of this biomarker in the urine sample of the prostate cancer patient compared to the reference value is typically from 101% to 300%, preferably from 105% to 200%.

In one embodiment, the cancer is prostate cancer, the biomarker has an m/z of 126 and a H⁺ pseudo molecular ion of m/z of 127 and a decrease in the level of this biomarker in the urine sample compared to the reference value is indicative of prostate cancer in the subject. In this embodiment, the biomarker is preferably 3-octen-2-one. In this embodiment, the % mean difference in the level of this biomarker in the urine sample of the prostate cancer patient compared to the reference value is typically from 110% to 500%, preferably from 120% to 250%.

In one embodiment, the cancer is prostate cancer, the biomarker has an m/z of 114 and a H⁺ pseudo molecular ion of m/z of 115 and a decrease in the level of this biomarker in the urine sample compared to the reference value is indicative of prostate cancer in the subject. In this embodiment, the biomarker is preferably 2-heptanone. In this embodiment, the % mean difference in the level of this biomarker in the urine sample of the prostate cancer patient compared to the reference value is typically from 110% to 500%, preferably from 120% to 250%.

In one embodiment, the cancer is prostate cancer, the biomarker has an m/z of 114 and a H⁺ pseudo molecular ion of m/z of 115 and a decrease in the level of this biomarker in the urine sample compared to the reference value is indicative of prostate cancer in the subject. In this embodiment, the biomarker is preferably 4-heptanone. In this embodiment, the % mean difference in the level of this biomarker in the urine sample of the prostate cancer patient compared to the reference value is typically from 105% to 400%, preferably from 110% to 200%.

In one embodiment, the cancer is renal cancer, the biomarker has an m/z of 186 and a H⁺ pseudo molecular ion of m/z of 187 and an increase in the level of this biomarker in the urine sample compared to the reference value is indicative of renal cancer in the subject. In this embodiment, the biomarker is preferably 1-dodecanol. In this embodiment, the % mean difference in the level of this biomarker in the urine sample of the renal cancer patient compared to the reference value is typically from 120% to 500%, preferably from 150% to 300%.

In one embodiment, the cancer is renal cancer, the biomarker has an m/z of 32 and a H⁺ pseudo molecular ion of m/z of 33 and a decrease in the level of this biomarker in the urine sample compared to the reference value is indicative of renal cancer in the subject. In this embodiment, the biomarker is preferably methanol. In this embodiment, the % mean difference in the level of this biomarker in the urine sample of the renal cancer patient compared to the reference value is typically from 110% to 200%, preferably from 120% to 160%.

In one embodiment, the cancer is renal cancer, the biomarker has an m/z of 81 and a H⁺ pseudo molecular ion of m/z of 82 and a decrease in the level of this biomarker in the urine sample compared to the reference value is indicative of renal cancer in the subject. In this embodiment, the biomarker is preferably 2-methyl-2-butenenitrile. In this embodiment, the % mean difference in the level of this biomarker in the urine sample of the renal cancer patient compared to the reference value is typically from 120% to 500%, preferably from 200% to 350%.

In one embodiment, the cancer is renal cancer, the biomarker has an m/z of 156 and a H⁺ pseudo molecular ion of m/z of 157 and a decrease in the level of this biomarker in the urine sample compared to the reference value is indicative of renal cancer in the subject. In this embodiment, the biomarker is preferably menthol. In this embodiment, the % mean difference in the level of this biomarker in the urine sample of the renal cancer patient compared to the reference value is typically from 1000% to 10,000%, preferably from 2000% to 6000%.

In one embodiment, the cancer is renal cancer, the biomarker has an m/z of 152 and a H⁺ pseudo molecular ion of m/z of 153 and a decrease in the level of this biomarker in the urine sample compared to the reference value is indicative of renal cancer in the subject. In this embodiment, the biomarker is preferably piperitone. In this embodiment, the % mean difference in the level of this biomarker in the urine sample of the renal cancer patient compared to the reference value is typically from 120% to 500%, preferably from 150% to 300%.

In one embodiment, the cancer is renal cancer, the biomarker has an m/z of 206 and a H⁺ pseudo molecular ion of m/z of 207 and a decrease in the level of this biomarker in the urine sample compared to the reference value is indicative of renal cancer in the subject. In this embodiment, the biomarker is preferably 2,4-bis(1,1-dimethylethyl)phenol. In this embodiment, the % mean difference in the level of this biomarker in the urine sample of the renal cancer patient compared to the reference value is typically from 150% to 1500%, preferably from 200% to 600%.

In one embodiment, the cancer is renal cancer, the biomarker has an m/z of 130 and a H⁺ pseudo molecular ion of m/z of 131 and an increase in the level of this biomarker in the urine sample compared to the reference value is indicative of renal cancer in the subject. In this embodiment, the biomarker is preferably 2-ethyl-1-hexanol. In this embodiment, the % mean difference in the level of this biomarker in the urine sample of the renal cancer patient compared to the reference value is typically from 120% to 800%, preferably from 140% to 400%.

In one embodiment, the cancer is renal cancer, the biomarker has an m/z of 142 and a H⁺ pseudo molecular ion of m/z of 143 and an increase in the level of this biomarker in the urine sample compared to the reference value is indicative of renal cancer in the subject. In this embodiment, the biomarker is preferably nonanal. In this embodiment, the % mean difference in the level of this biomarker in the urine sample of the renal cancer patient compared to the reference value is typically from 101% to 300%, preferably from 105% to 200%.

In one embodiment, the cancer is renal cancer, the biomarker has an m/z of 98 and a H⁺ pseudo molecular ion of m/z of 99 and an increase in the level of this biomarker in the urine sample compared to the reference value is indicative of renal cancer in the subject. In this embodiment, the biomarker is preferably cyclohexanone. In this embodiment, the % mean difference in the level of this biomarker in the urine sample of the renal cancer patient compared to the reference value is typically from 150% to 1000%, preferably from 200% to 500%.

In one embodiment, the cancer is bladder cancer, the biomarker has an m/z of 32 and a H⁺ pseudo molecular ion of m/z of 33 and an increase in the level of this biomarker in the urine sample compared to the reference value is indicative of bladder cancer in the subject. In this embodiment, the biomarker is preferably methanol. In this embodiment, the % mean difference in the level of this biomarker in the urine sample of the bladder cancer patient compared to the reference value is typically from 200% to 800%, preferably from 300% to 500%.

In one embodiment, the cancer is bladder cancer, the biomarker has an m/z of 72 and a H⁺ pseudo molecular ion of m/z of 73 and an increase in the level of this biomarker in the urine sample compared to the reference value is indicative of bladder cancer in the subject. In this embodiment, the biomarker is preferably 2-butanone. In this embodiment, the % mean difference in the level of this biomarker in the urine sample of the bladder cancer patient compared to the reference value is typically from 110% to 250%, preferably from 120% to 200%.

In one embodiment, the cancer is bladder cancer, the biomarker has an m/z of 46 and a H⁺ pseudo molecular ion of m/z of 47 and a decrease in the level of this biomarker in the urine sample compared to the reference value is indicative of bladder cancer in the subject. In this embodiment, the biomarker is preferably ethanol. In this embodiment, the % mean difference in the level of this biomarker in the urine sample of the bladder cancer patient compared to the reference value is typically from 200% to 2000%, preferably from 600% to 1000%.

In one embodiment, the cancer is bladder cancer, the biomarker has an m/z of 156 and a H⁺ pseudo molecular ion of m/z of 157 and a decrease in the level of this biomarker in the urine sample compared to the reference value is indicative of bladder cancer in the subject. In this embodiment, the biomarker is preferably menthol. In this embodiment, the % mean difference in the level of this biomarker in the urine sample of the bladder cancer patient compared to the reference value is typically from 500% to 5000%, preferably from 1500% to 3500%.

In one embodiment, the cancer is bladder cancer, the biomarker has an m/z of 130 and a H⁺ pseudo molecular ion of m/z of 131 and an increase in the level of this biomarker in the urine sample compared to the reference value is indicative of bladder cancer in the subject. In this embodiment, the biomarker is preferably 2-ethyl-1-hexanol. In this embodiment, the % mean difference in the level of this biomarker in the urine sample of the bladder cancer patient compared to the reference value is typically from 101% to 150%, preferably from 103% to 130%.

In one embodiment, the cancer is bladder cancer, the biomarker has an m/z of 142 and a H⁺ pseudo molecular ion of m/z of 143 and an increase in the level of this biomarker in the urine sample compared to the reference value is indicative of bladder cancer in the subject. In this embodiment, the biomarker is preferably nonanal. In this embodiment, the % mean difference in the level of this biomarker in the urine sample of the bladder cancer patient compared to the reference value is typically from 105% to 250%, preferably from 110% to 200%.

In one embodiment, the cancer is bladder cancer, the biomarker has an m/z of 114 and a H⁺ pseudo molecular ion of m/z of 115 and a decrease in the level of this biomarker in the urine sample compared to the reference value is indicative of bladder cancer in the subject. In this embodiment, the biomarker is preferably 2-heptanone. In this embodiment, the % mean difference in the level of this biomarker in the urine sample of the bladder cancer patient compared to the reference value is typically from 105% to 200%, preferably from 110% to 150%.

In one embodiment, the cancer is bladder cancer, the biomarker has an m/z of 114 and a H⁺ pseudo molecular ion of m/z of 115 and a decrease in the level of this biomarker in the urine sample compared to the reference value is indicative of bladder cancer in the subject. In this embodiment, the biomarker is preferably 4-heptanone. In this embodiment, the % mean difference in the level of this biomarker in the urine sample of the bladder cancer patient compared to the reference value is typically from 120% to 500%, preferably from 130% to 300%.

In one embodiment, the cancer is colorectal cancer, the biomarker has an m/z of 94 and a H⁺ pseudo molecular ion of m/z of 95 and an increase in the level of this biomarker in the urine sample compared to the reference value is indicative of bladder cancer in the subject. In this embodiment, the biomarker is preferably phenol. In this embodiment, the % mean difference in the level of this biomarker in the urine sample of the colorectal cancer patient compared to the reference value is typically from 500% to 10,000%, preferably from 1000% to 5000%.

In one embodiment, the cancer is colorectal cancer, the biomarker has an m/z of 98 and a H⁺ pseudo molecular ion of m/z of 99 and an increase in the level of this biomarker in the urine sample compared to the reference value is indicative of bladder cancer in the subject. In this embodiment, the biomarker is preferably cyclohexanone. In this embodiment, the % mean difference in the level of this biomarker in the urine sample of the colorectal cancer patient compared to the reference value is typically from 500% to 10,000%, preferably from 1000% to 5000%.

In one embodiment, the cancer is colorectal cancer, the biomarker has an m/z of 130 and a H⁺ pseudo molecular ion of m/z of 131 and an increase in the level of this biomarker in the urine sample compared to the reference value is indicative of bladder cancer in the subject. In this embodiment, the biomarker is preferably 2-ethyl-1-hexanol. In this embodiment, the % mean difference in the level of this biomarker in the urine sample of the colorectal cancer patient compared to the reference value is typically from 300% to 5000%, preferably from 500% to 1000%.

In one embodiment, the cancer is colorectal cancer, the biomarker has an m/z of 120 and a H⁺ pseudo molecular ion of m/z of 121 and an increase in the level of this biomarker in the urine sample compared to the reference value is indicative of bladder cancer in the subject. In this embodiment, the biomarker is preferably acetophenone. In this embodiment, the % mean difference in the level of this biomarker in the urine sample of the colorectal cancer patient compared to the reference value is typically from 150% to 700%, preferably from 1000% to 5000%.

In one embodiment, the cancer is colorectal cancer, the biomarker has an m/z of 114 and a H⁺ pseudo molecular ion of m/z of 115 and an increase in the level of this biomarker in the urine sample compared to the reference value is indicative of bladder cancer in the subject. In this embodiment, the biomarker is preferably butyl acetata. In this embodiment, the % mean difference in the level of this biomarker in the urine sample of the colorectal cancer patient compared to the reference value is typically from 120% to 1000%, preferably from 200% to 600%.

In one embodiment, the cancer is colorectal cancer, the biomarker has an m/z of 58 and a H⁺ pseudo molecular ion of m/z of 59 and an increase in the level of this biomarker in the urine sample compared to the reference value is indicative of bladder cancer in the subject. In this embodiment, the biomarker is preferably acetone. In this embodiment, the % mean difference in the level of this biomarker in the urine sample of the colorectal cancer patient compared to the reference value is typically from 110% to 300%, preferably from 120% to 200%.

In one embodiment, the cancer is colorectal cancer, the biomarker has an m/z of 114 and a H⁺ pseudo molecular ion of m/z of 115 and a decrease in the level of this biomarker in the urine sample compared to the reference value is indicative of bladder cancer in the subject. In this embodiment, the biomarker is preferably acetone. In this embodiment, the % mean difference in the level of this biomarker in the urine sample of the colorectal cancer patient compared to the reference value is typically from 120% to 250%, preferably from 150% to 200%.

It will be seriously contemplated by the skilled person that the increases and/or decreases of the various specific biomarkers referred to in the above embodiments may be combined in any or all combinations for each cancer such that the combinations of the increase and/or decrease of each biomarker (preferably in the typical and preferred % mean differences specified above) represents a particularly preferred “fingerprint” for that particular cancer. In particular, the following preferred combinations of the levels of the following biomarkers in urine sample compared to reference values are particularly indicative of the specified cancer in the subject:

Pancreatic cancer—any combination of any or all of the following:

Increase: octanal, heptanal, nonanal, 2-octanone, 2-nonanone and/or 2-decanone; and/or

Decrease: an ethylbenzaldehyde or a dimethylbenzaldehyde; 1-dodecanol, 2,4-bis(1,1-dimethylethyl)phenol; menthol; 2-methyl-2-butenenitrile, 2-heptanone and/or 4-heptanone.

Lung cancer—any combination of any or all of the following:

Increase: octanal, nonanal, 2-octanone, 2,6-dimethylpyrazine N-methylmethanamine and/or benzene; and/or

Decrease: an ethylbenzaldehyde or a dimethylbenzaldehyde; 1-dodecanol, 2,4-bis(1,1-dimethylethyl)phenol; 3-ethyl[1,2,4]triazolo[4,3-a]pyridine; oxygenated sesquiterpene; 2-pentanone, 2-ethyl-1-hexanol and/or 2-heptanone.

Breast cancer—any combination of any or all of the following:

Increase: 3-ethylcyclopentanone; piperitone and/or acetone; and/or

Decrease: an ethylbenzaldehyde or a dimethylbenzaldehyde; 1-dodecanol; 3-ethyl[1,2,4]triazolo[4,3-a]pyridine; 1-tetradecene, and/or 4-heptanone.

Prostate cancer:

Increase: oxygenated sesquiterpene, menthol, 2,4-bis(1,1-dimethylethyl)phenol and/or nonanal;

and/or

Decrease: an ethylbenzaldehyde or a dimethylbenzaldehyde; ethanol; acetone; 1-dodecanol, 3-octen-2-one, 2-heptanone and/or 4-heptanone.

Renal cancer:

Increase: 1-dodecanol, 2-ethyl-1-hexanol, nonanal and/or cyclohexanone;

and/or

Decrease: an ethylbenzaldehyde or a dimethylbenzaldehyde; methanol; 2-methyl-2-butenenitrile; oxygenated sesquiterpene (menthol); oxygenated sesquiterpene (piperitone); and/or 2,4-bis(1,1-dimethylethyl)phenol.

Bladder cancer:

Increase: methanol, 2-butanone, 2-ethyl-1-hexanol and/or nonanal;

and/or

Decrease: an ethylbenzaldehyde or a dimethylbenzaldehyde; ethanol, methanol; menthol and/or 4-heptanone.

Colorectal cancer:

Increase: phenol; cyclohexanone; 2-ethyl-1-hexanol, acetophenone, butyl acetate and/or acetone;

and/or

Decrease: 4-heptanone.

Uses, Applications and Methods of Treatment

The method of the present invention may be used to identify patients having cancer. Patients identified using the method may subsequently be treated for cancer.

Therefore, in a further aspect, the invention provides a substance for use in a method of treating cancer in a subject, where the subject is identified as requiring treatment by the method of the invention.

In a yet further aspect, the invention provides use of a substance in the manufacture of a medicament for treating cancer in a subject, where the subject is identified as requiring treatment by the method of the invention.

In a still further aspect, the invention provides a method of treating cancer in a subject, the method comprising:

(a) identifying a subject requiring treatment by the method of the invention; and

(b) administering an anti-cancer agent to the subject requiring treatment.

The treatment may be carried out using any substance known in the art as an anti-cancer agent. Examples of anticancer agents are well known to those skilled in the art.

For instance, the anticancer agent may comprise a chemotherapeutic agent. A “chemotherapeutic agent” is a biological (large molecule) or chemical (small molecule) compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, proteins, antibodies, photosensitizers, and kinase inhibitors. Chemotherapeutic agents include compounds used in “targeted therapy” and non-targeted, conventional chemotherapy.

Examples of chemotherapeutic agents include thioTEPA, doxorubicin, vincristine, rituximab, cyclophosphamide, prednisone, melphalan, lenalidomide, bortezomib, rapamycin, and cytarabine.

Examples of chemotherapeutic agents also include: erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diaminedichloroplatinum(II), CAS No. 15663-27-1), carboplatin (CAS No. 41575-94-4), paclitaxel (TAXOL®, Bristol-Myers Squibb Oncology), temozolomide (4-methyl-5-oxo-2,3,4,6,8-pentazabicyclo [4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethyl-ethanamine, NOLVADEX®, ISTUBAL®, VALODEX®), doxorubicin (ADRIAMYCIN®), Akti-1/2, HPPD, rapamycin, and lapatinib (TYKERB®, GlaxoSmithKline).

More examples of chemotherapeutic agents include: oxaliplatin (ELOXATIN®, Sanofi), bortezomib (VELCADE®, Millennium Pharm.), sunitinib (SUTENT®, SU11248, Pfizer), letrozole (FEMARA®, Novartis), imatinib mesylate (GLEEVEC®, Novartis), XL-518 (MEK inhibitor, Exelixis, WO 2007/044515), ARRY-886 (MEK inhibitor, AZD6244, Array BioPharma, AstraZeneca), SF-1126 (PI3K inhibitor, Semafore Pharmaceuticals), BEZ-235 (PI3K inhibitor, Novartis), XL-147 (PI3K inhibitor, Exelixis), ABT-869 (multi-targeted inhibitor of VEGF and PDGF family receptor tyrosine kinases, Abbott Laboratories and Genentech), ABT-263 (Bc1-2/Bcl-xL inhibitor, Abbott Laboratories and Genentech), PTK787/ZK 222584 (Novartis), fulvestrant (FASLODEX®, AstraZeneca), leucovorin (folinic acid), lonafamib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), capecitabine (XELODA®, Roche), ABRAXANE™ (Cremophor-free), albumin-engineered nanoparticle formulations of paclitaxel (American Pharmaceutical Partners, Schaumberg, 11), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thioTepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chiorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, calicheamicin gamma II, calicheamicin omega II, dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; antiadrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; tiaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thioTepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; ibandronate; CPT-11; topoisomerase inhibitor RFS 2000; difluoromethylomithine (DMFO); retinoids such as retinoic acid; and pharmaceutically acceptable salts, acids and derivatives of any of the above.

Also included in the definition of “chemotherapeutic agent” are: (i) antihormonal agents that act to regulate or inhibit hormone action on tumors such as antiestrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKN® rIL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTN®, Genentech); cetuximab (ERBITUX®, Imclone); panitumumab (VECTIBIX®, Amgen), rituximab (RITUXAN®, Genentech/Biogen Idec), pertuzumab (OMNITARG™, rhuMab 2C4, Genentech), trastuzumab (HERCEPTIN®, Genentech), tositumomab (Bexxar, Corixia), and the antibody drug conjugate, gemtuzumab ozogamicin (MYLOTARG®, Wyeth).

Humanised monoclonal antibodies with therapeutic potential as chemotherapeutic agents in combination with the PI3K inhibitors of the invention include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motovizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, rolizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

In one embodiment, the anti-cancer agent is a biomarker identified by a method according to the invention. Without wishing to be bound by theory, it is believed that one or more of the biomarkers identified using the claimed methods may be involved in the metabolic pathway of the cancer. The method therefore confers the potential for a method of treatment using the specified biomarker in order to reverse this step in the pathway and treat the cancer. In one embodiment, the method is a supplementation of the specified biomarker (in the case where a decrease in the level of this biomarker in the urine sample compared to the reference value is indicative of cancer in the subject). In another embodiment, the method is a deprivation of the specified biomarker (in the case where an increase in the level of this biomarker in the urine sample compared to the reference value is indicative of cancer in the subject).

In particularly preferred embodiments, the invention provides an ethylbenzaldehyde or a dimethylbenzaldehyde for use in a method of treating cancer in a subject, where the subject is identified as requiring treatment by a method of the invention. In some embodiments, the methods provide supplementation of an ethylbenzaldehyde or a dimethylbenzaldehyde (in the case where a decrease in the level of this biomarker in the urine sample compared to the reference value is indicative of cancer in the subject).

Typically, the method is carried out by administering the anti-cancer agent in the form of a pharmaceutical composition. Examples of pharmaceutical compositions are known to those skilled in the art.

The anti-cancer agent may be administered orally, intravenously, subcutaneously, buccally, rectally, dermally, nasally, tracheally, bronchially, sublingually, by any other parenteral route or via inhalation, in a pharmaceutically acceptable dosage form.

The anti-cancer agent may be administered alone, but are preferably administered by way of known pharmaceutical formulations, including tablets, capsules or elixirs for oral administration, suppositories for rectal administration, sterile solutions or suspensions for parenteral or intramuscular administration, and the like. The type of pharmaceutical formulation may be selected with due regard to the intended route of administration and standard pharmaceutical practice. Such pharmaceutically acceptable carriers may be chemically inert to the active compounds and may have no detrimental side effects or toxicity under the conditions of use.

Such formulations may be prepared in accordance with standard and/or accepted pharmaceutical practice. Otherwise, the preparation of suitable formulations may be achieved non-inventively by the skilled person using routine techniques and/or in accordance with standard and/or accepted pharmaceutical practice.

The anti-cancer agent may be administered in admixture with a pharmaceutically acceptable adjuvant, diluent and/or carrier. Depending on e.g. potency and physical characteristics of the active ingredient, pharmaceutical formulations that may be mentioned include those in which the active ingredient is present in at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

The amount of the anti-cancer agent in the formulation will depend on the severity of the condition, and on the patient, to be treated, as well as the compound(s) which is/are employed, but may be determined non-inventively by the skilled person.

### EXAMPLES

### Example 1

The volatile components in urine samples were identified by GCMS, using a volatile entrainment technique known as Solid-Phase MicroExtraction. The urine samples (0.2 ml) were first treated with alkali (0.2 ml 1M NaOH) and equilibrated for 30 min at 50° C. and the headspace volatiles are injected onto a special thick film column (Supleco, SPB-1 30 m×0.32 mm, 4 μm film) and analysed by full scan MS using an HP 5890 series II GC-MS device under the following conditions:

(1) Injector port 1 mm SPME injector linear heated to 220° C.;

(2) Column oven 40° C. (2 mins) ramped by 10° C./min to 240° C. and held at these conditions for 4 mins; and

(3) Analysed by HP5971 mass spectrometer scanned over the range 29-450 amu (m/z) at 2 scans/second using the electron impact (El) method.

The GCMS data was processed by visual comparisons of data set chromatograms and identifying differences using the NIST MS library, and by advanced biomarker and metabolomic profiling open-source software, known as XCMS (Scripps Institute, La Jolla, Calif.). The XCMS software provides a way of combining all the GCMS data into sets and show differences between the mass ion(s)s and retention times in the data sets. The software also provides the statistical importance (p-Value and Fold values) of each mass ion(s) difference across the GCMS chromatogram and confirms if the mass ion(s) is up or down regulated compared to the controls.

The XCMS software was used to calculate the % mean difference. This software provides a method in which each mass ion, across a time or chromatographic window, is summed for a given mass spec analysed sample sets. Summed sample sets (i.e. controls and cancers) are compared and a % mean difference of mass ions at any given time or chromatographic window is produced. Any representative ion showing a significant % mean difference can be used at a particular time or chromatographic window to help provide “biomarker identification” when using the original sample mass spec data.

Bladder, renal, prostate, pancreatic, lung and breast cancer urine samples have been analysed and compared with non-cancer (control) urine samples, on the following numbers of samples:

Bladder cancer: 11 cancer patients; 11 controls.

Renal cancer: 10 cancer patients; 10 controls.

Prostate cancer: 5 cancer patients; 6 controls.

Breast cancer: 5 cancer patients; 6 controls.

Lung cancer: 5 cancer patients; 6 controls.

Pancreatic cancer: 4 cancer patients; 6 controls.

The results are shown in the Tables below—bladder cancer (Table 1), renal cancer (Table 2), prostate cancer (Table 3), breast cancer (Table 4), lung cancer (Table 5) and pancreatic cancer (Table 6). In the Tables, where an upregulation was observed (“Up”) (i.e. the measurement comprises an increase of the total ion count for the appropriate mass ion(s) in subjects having cancer, compared with the reference value), the % mean difference is measured as (mean cancer/mean control)×100%.

Where a downregulation was observed (“Down”) (i.e. the measurement comprises a decrease of the total ion count for the appropriate mass ion(s) in subjects having cancer, compared with the reference value), the % mean difference is measured as (mean control/mean cancer)×100%.

The m/z of the mass ion(s) referred to in the Table below may be that of the molecular ion (M) or that of a fragment ion (F) which is characteristic of the parent ion. The compounds were identified either by the molecular ion or, for fragment ions, by the presence of that fragment ion in a reference mass spectrum for that compound. Reference spectra can be found, for example, in the NIST MS library http://chemdata.nist.gov/. Ethylbenzaldehyde or dimethylbenzaldehyde were identified by molecular ion (M+1) by means of a ¹³C isotope. Representative m/z ions used to show significant differences between sample sets have been used in these examples, and are not limited to these example m/z ions.

TABLE 1

| | | | | Reten- | |
|---|---|---|---|---|---|
| | | | | tion | |
| | Up/ | | | Time | % mean |
| Compound | down | m/z | | (min) | diff |
| Methanol | Up | 31 | (F) | 2.30 | 373 |
| 2-Butanone | Up | 72 | (M) | 5.83 | 158 |
| Ethanol | Down | 31 | (F) | 3.04 | 753 |
| Menthol | Down | 81 | (F) | 20.51 | 2647 |
| Ethyl Benzaldehyde or | Down | 135 | (M + 1) | 21.18 | 340 |
| Dimethyl Benzaldehyde | | | | | |

TABLE 2

| | | | | Reten- | |
|---|---|---|---|---|---|
| | | | | tion | |
| | Up/ | | | Time | % mean |
| Compound | down | m/z | | (min) | diff |
| 1-dodecanol | Up | 111 | (F) | 25.64 | 217 |
| Methanol | Down | 31 | (F) | 2.31 | 144 |
| 2-Methyl-2-Butenenitrile | Down | 81 | (M) | 10.30 | 233 |
| Menthol | Down | 81 | (F) | 20.50 | 4011 |
| Ethyl Benzaldehyde or | Down | 135 | (M + 1) | 21.17 | 376 |
| Dimethyl Benzaldehyde | | | | | |
| Piperitone | Down | 110 | (F) | 21.98 | 208 |
| 2,4-bis (1,1-dimethylethyl)- | Down | 191 | (F) | 26.28 | 433 |
| phenol | | | | | |

TABLE 3

| | | | | Reten- | |
|---|---|---|---|---|---|
| | | | | tion | |
| | Up/ | | | Time | % mean |
| Compound | down | m/z | | (min) | diff |
| Oxygenated sesquiterpine | Up | 150 | (F) | 21.74 | 2011 |
| Menthol | Up | 81 | (F) | 20.52 | 559 |
| 2,4-bis(1,1-dimethylethyl)- | Up | 191 | (F) | 26.28 | 160 |
| phenol | | | | | |
| Ethanol | Down | 31 | (F) | 3.03 | 210 |
| Acetone | Down | 58 | (M) | 3.54 | 212 |
| Ethyl Benzaldehyde or | Down | 135 | (M + 1) | 21.16 | 1350 |
| Dimethyl Benzaldehyde | | | | | |
| 1-dodecanol | Down | 111 | (F) | 25.63 | 2224 |

TABLE 4

| | | | | Reten- | |
|---|---|---|---|---|---|
| | | | | tion | |
| | Up/ | | | Time | % mean |
| Compound | down | m/z | | (min) | diff |
| 3-Ethylcyclopentanone | Up | 83 | (F) | 15.35 | 215 |
| Piperitone | Up | 110 | (F) | 21.99 | 314 |
| Ethyl Benzaldehyde or | Down | 135 | (M + 1) | 21.16 | 1241 |
| Dimethyl Benzaldehyde | | | | | |
| 1-Dodecanol | Down | 111 | (F) | 25.65 | 1718 |
| 3-ethyl[1,2,4]triazolo[4,3- | Down | 146 | (F) | 22.63 | 3647 |
| a]pyridine | | | | | |
| 1-Tetradecene | Down | 97 | (F) | 25.65 | 1604 |

TABLE 5

| | | | | Reten- | |
|---|---|---|---|---|---|
| | | | | tion | |
| | Up/ | | | Time | % mean |
| Compound | down | m/z | | (min) | diff |
| 2,4-bis(1,1-Dimethyl- | Down | 191 | (F) | 26.27 | 372 |
| ethyl)-phenol | | | | | |
| Ethyl Benzaldehyde or | Down | 135 | (M + 1) | 21.15 | 1613 |
| Dimethyl Benzaldehyde | | | | | |
| 1-Dodecanol | Down | 111 | (F) | 25.65 | 1718 |
| 3-ethyl[1,2,4]triazolo[4,3- | Down | 146 | (F) | 22.59 | 2694 |
| a]pyridine | | | | | |
| Oxygenated | Down | 81.1 | (F) | 20.50 | 103 |
| sesquiterpine | | | | | |
| 2-Pentanone | Down | 86 | (M) | 8.215 | 178 |
| Nonanal | Up | 96. | (F) | 18.69 | 1892 |
| 2-Nonanone | Up | 58. | (M) | 18.39 | 2249 |
| Octanal | Up | 56 | (M) | 16.40 | 1930 |
| 2,5-Dimethylpyrazine | Up | 108 | (M) | 14.19 | 232 |
| N-Methyl-methanamine | Up | 45 | (M) | 2.68 | 142 |

TABLE 6

| | | | | Reten- | |
|---|---|---|---|---|---|
| | | | | tion | |
| | Up/ | | | Time | % mean |
| Compound | down | m/z | | (min) | diff |
| Ethyl Benzaldehyde or | Down | 135 | (M + 1) | 21.16 | 12867 |
| Dimethyl Benzaldehyde | | | | | |
| 1-Dodecanol | Down | 111 | (F) | 25.61 | 3859 |
| 2,4-bis(1,1- | Down | 191 | (F) | 26.27 | 290 |
| Dimethylethyl)-phenol | | | | | |
| Menthol | Down | 81 | (F) | 20.50 | 736 |
| 2-Methyl-2-butenenitrile | Down | 81 | (F) | 10.28 | 12240 |
| Octanal | Up | 56 | (F) | 16.43 | 1142 |
| Heptanal | Up | 70 | (F) | 13.98 | 789 |
| Nonanal | Up | 96 | (F) | 18.68 | 401 |
| 2-Nonanone | Up | 58 | (F) | 18.38 | 3266 |
| 2-Decanone | Up | 58 | (F) | 20.51 | 5835 |
| 2-Octanone | Up | 58 | (F) | 16.12 | 979 |

### Example 2

The volatile components in another set of urine samples from the patient groups listed below were identified by GCMS using the same general method as taught in example 1.

The volatile components in urine samples were identified by GCMS, using a volatile entrainment technique known as Solid-Phase MicroExtraction. The urine samples (0.2 ml) were first treated with alkali (0.2 ml 1M NaOH) and equilibrated for 30 min at 50° C. and the headspace volatiles are injected onto a special thick film column (Supleco, SPB-1 30 m×0.32 mm, 4 μm film) and analysed by full scan MS using an HP 5890 series II GC-MS device under the following conditions:

(1) Injector port 1 mm SPME injector linear heated to 220° C.;

(2) Column oven 40° C. (2 mins) ramped by 10° C./min to 240° C. and held at these conditions for 4 mins; and

(3) Analysed by HP5971 mass spectrometer scanned over the range 29-450 amu (m/z) at 2 scans/second using the electron impact (El) method.

The GCMS data was processed by visual comparisons of data set chromatograms and identifying differences using the NIST MS library, and by advanced biomarker and metabolomic profiling open-source software, known as XCMS (Scripps Institute, La Jolla, Calif.). The XCMS software provides a way of combining all the GCMS data into sets and show differences between the mass ion(s)s and retention times in the data sets. The software also provides the statistical importance (p-Value and Fold values) of each mass ion(s) difference across the GCMS chromatogram and confirms if the mass ion(s) is up or down regulated compared to the controls.

The XCMS software was used to calculate the % mean difference. This software provides a method in which each mass ion, across a time or chromatographic window, is summed for a given mass spec analysed sample sets. Summed sample sets (i.e. controls and cancers) are compared and a % mean difference of mass ions at any given time or chromatographic window is produced. Any representative ion showing a significant % mean difference can be used at a particular time or chromatographic window to help provide “biomarker identification” when using the original sample mass spec data.

Bladder, renal, prostate, pancreatic, lung, colorectal and breast cancer urine samples have been analysed and compared with non-cancer (control) urine samples, on the following numbers of samples:

Bladder cancer: 6 cancer patients; 6 controls.

Renal cancer: 9 cancer patients; 6 controls.

Prostate cancer: 4 cancer patients; 6 controls.

Breast cancer: 9 cancer patients; 6 controls.

Lung cancer: 10 cancer patients; 6 controls.

Pancreatic cancer: 8 cancer patients; 6 controls.

Colorectal cancer: 10 cancer patients; 6 controls

The volatile components in urine samples can also be identified by SESI MS, using a volatile entrainment technique known as Solid-Phase MicroExtraction. In a similar manner to example 1, the urine samples (0.2 ml) can be first treated with alkali (0.2 ml 1M NaOH) and equilibrated for 30 min at 50° C. The headspace volatiles can then be desorbed via resistive heater in the immediate vicinity and analysed by full scan MS and MSⁿ data dependant scanning using an LCQ DECA ion trap mass spectrometer with nano electrospray within the electrospray ionization chamber:

(1) Desorption Heater heated to 220° C.;

(2) Nano Spray of 0.1% formic acid (v/v) in deionised water (18 MΩ) as the electrospray solution, delivered at 5 nL/s through a non-conductive silica capillary with a sharpened needle tip (40 μm ID). The applied voltage is 3.5 kV.

(3) Desolvation Capillary Temperature 180° C.

(4) Analysed mass spectrometer scanned over the range 29-450 amu (m/z) at 2 scans/second with automatic data dependent scanning (29-450 amu).

The SESI-MS data can then be processed by advanced biomarker and metabolomic profiling open-source software, known as XCMS (Scripps Institute, La Jolla, Calif.). The XCMS software provides a way of combining all the SESI-MS data into sets and show differences between the mass ion(s) and retention times in the data sets. The software also provides the statistical importance (p-Value and Fold values) of each mass ion(s) difference across the SESI-MS total ion chromatograms and confirms if the mass ion(s) is up or down regulated compared to the controls.

The XCMS software can then be used to calculate the % mean difference. This software provides a method in which each mass ion, across a time or chromatographic window, is summed for a given mass spec analysed sample sets. Summed sample sets (i.e. controls and cancers) are compared and a % mean difference of mass ions at any given time or chromatographic window is produced. Any representative ion showing a significant % mean difference can be used at a particular time or chromatographic window to help provide “biomarker identification” when using the original sample mass spec data.

The results are shown in Table 7.

TABLE 7

| | | % Mean Diff | | | | | |
|---|---|---|---|---|---|---|---|
| | | Up/Down | | | GCMS | *SESI | *SESI MS2 |
| Cancer | Compound | Regulated | | GCMS ions | r.t. | M + H ion | qualifier ion |
| PROSTATE | Nonanal | 117% | up | 98/95/82/57 | 18.68 | 143 | 98/57/43 |
| | 3-Octen-2-one | 141% | down | 111/55 | 17.10 | 127 | 111 |
| | 2-Heptanone | 134% | down | 58/43 | 13.61 | 115 | 58/43 |
| | 4-Heptanone | 121% | down | 71/43 | 13.24 | 115 | 71 |
| RENAL | 2-Ethyl-1- | 180% | up | 57/70/82 | 17.05 | 129 | 57/70/82 |
| | Hexanol | | | | | | |
| | Nonanal | 118% | up | 98/95/82/57 | 18.68 | 143 | 98/57/43 |
| | Cyclohexanone | 350% | up | 98/80/42 | 13.77 | 99 | 80 |
| PANCREATIC | Nonanal | 117% | up | 96/95/82 | 18.68 | 145 | 96 |
| | 2-Heptanone | 127% | down | 58/43 | 13.61 | 115 | 58/43 |
| | 4-Heptanone | 118% | down | 71/43 | 13.24 | 115 | 71 |
| LUNG | Benzene | 414% | up | 78 | 7.31 | 79 | |
| | 2-Ethyl-1- | 126% | up | 57/70/82 | 17.05 | 129 | 57/70/82 |
| | Hexanol | | | | | | |
| | 2-Heptanone | 106% | down | 58/43 | 13.61 | 115 | 58/43 |
| BREAST | Acetone | 110% | up | 58/3 | 3.55 | 59 | 43 |
| | 4-Heptanone | 127% | down | 71/43 | 13.24 | 115 | 71 |
| COLORECTAL | Phenol | 1500% | up | 94/66 | 15.56 | 95 | 66/65 |
| | Cyclohexanone | 1445% | up | 98/69/55/42 | 13.75 | 99 | 69/55 |
| | 2-Ethyl-1- | 993% | up | 57/70/82 | 17.05 | 129 | 57/70/82 |
| | Hexanol | | | | | | |
| | Acetophenone | 580% | up | 120/105/77 | 17.87 | 121 | 105/77 |
| | Butyl Acetate | 333% | up | 61/43 | 11.70 | 117 | 61/56/43 |
| | Acetone | 130% | up | 58/3 | 3.55 | 59 | 43 |
| | 4-Heptanone | 177% | down | 71/43 | 13.24 | 115 | 71 |
| BLADDER | 2-Ethyl-1- | 109% | up | 57/70/82 | 17.05 | 129 | 57/70/82 |
| | Hexanol | | | | | | |
| | Nonanal | 126% | up | 98/95/82/57 | 18.68 | 143 | 98/57/43 |
| | 2-Heptanone | 115% | down | 58/43 | 13.61 | 115 | 58/43 |
| | 4-Heptanone | 181% | down | 71/43 | 13.24 | 115 | 71 |

All publications mentioned in the above specification are herein incorporated by reference. Various modifications and variations of the described methods of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in chemistry, biochemistry and medicine or related fields are intended to be within the scope of the following claims.

1. A method of diagnosing cancer in a subject comprising determining a level of one or more volatile organic compounds in the headspace of a urine sample obtained from the subject, wherein the identity and/or level of the volatile organic compound is determined by tandem mass spectrometry.

2. A method according to claim 1, wherein the mass spectrometry is gas chromatography-mass spectrometry.

3. A method according to claim 1, wherein the tandem mass spectrometry is selected from ion trap mass spectrometry, quadrupole time-of-flight mass spectrometry, triple quadrupole mass spectrometry, Quadrupole Ion Trap mass spectrometry, Ion Mobility-Quadrupole Ion Trap-Time of Flight mass spectrometry, Quadrupole-Orbitrap mass spectrometry, Ion Mobility Spectrometer-Quadrupole Ion Trap mass spectrometry, Triple-Quadrupole-Orbitrap mass spectrometry, Quadrupole Ion Trap-Orbitrap mass spectrometry, Time of Flight or Ion Trap-Fourier Transform mass spectrometry.

4. A method according to claim 1, wherein the tandem mass spectrometry is secondary electrospray ionization mass spectrometry.

5. A method according to claim 1, wherein the volatile organic compound has a molecular mass of 32 to 290 u.

6. A method according to claim 1, wherein the urine sample is treated with an alkali prior to the mass spectrometry.

7. A method according to claim 6, wherein the urine sample which undergoes mass spectrometry analysis is treated with alkali such that the pH is between 9 and 14, preferably between 11 and 14, and most preferably between 13 and 14.

8. A method according to claim 1, wherein the urine sample headspace is exposed to an absorbent or absorbents prior to the mass spectrometry.

9. A method according to claim 8, wherein desorption is carried out prior to the mass spectrometry.

10. A method according to claim 1, further comprising comparing the level of the one or more volatile organic compounds in the urine sample to a reference value, wherein the level of the one or more volatile organic compounds in the urine sample compared to the reference value is indicative of cancer in the subject.

11. The method according to claim 10, wherein the level of the one or more volatile organic compounds is determined and a decrease in the level of the one or more volatile organic compounds in the urine sample compared to the reference value is indicative of cancer in the subject.

12. The method according to claim 10, wherein the level of the one or more volatile organic compounds is determined and an increase in the level of the one or more volatile organic compounds in the urine sample compared to the reference value is indicative of cancer in the subject.

13. A method according to claim 1, wherein the cancer is selected from bladder, renal, lung, prostate, pancreatic, breast or colorectal cancer.

14. (canceled)

15. (canceled)
